(19) United States
(12) Patent Application Publication
(10) Pub. No.: US 2024/0085426 A1
(43) Pub. Date: Mar. 14, 2024

(54) TRIPLE QUADRUPOLE-BASED MULTIPLE REACTION MONITORING FOR CLINICAL THERAPEUTIC RESPONSE PREDICTION IN CANCER

(71) Applicant: NATIONAL CANCER CENTER, Goyang-si (KR)

(72) Inventors: Hark Kyun KIM, Goyang-si (KR); Yu Ri CHOI, Goyang-si (KR)

(73) Assignee: NATIONAL CANCER CENTER, Goyang-si (KR)

(21) Appl. No.: 18/260,186

(22) PCT Filed: Nov. 22, 2021
(86) PCT No.: PCT/KR2021/017136

§ 371 Date: Jun. 30, 2023

(30) Foreign Application Priority Data
Dec. 30, 2020 (KR) 10-2020-0187190

(51) Int. Cl. G01N 33/574; C12Q 1/6886; G01N 33/68

(52) U.S. Cl. CPC G01N 33/57484; C12Q 1/6886; G01N 33/6848; C12Q 2600/158; G01N 2800/52

(57) ABSTRACT

The present invention relates to a method for predicting the reactivity of a cancer-targeted therapeutic agent, in which various types of target proteins targeted by a cancer-targeted therapeutic agent are simultaneously quantified through triple quadrupole mass spectrometry. The inventors of the present invention carefully selected peptide sequences with high selectivity in nanoflow liquid chromatography-triple quadrupole mass spectrometry, in response to clinical needs, and optimized peptide combinations and experimental conditions to enable multiple peptide quantification. As a result, validated predictive markers which can be used in cancer treatment or clinical trials, such as HER2, FGFR2, EGFR, MET, and PD-L1, and novel immunotherapy efficacy predictors (TAP2, I23O1 (IDO1), SYWC (WARS1), and UB2L6 (UBE2L6)) identified by the inventors of the present invention, can be quantified at the protein level simultaneously with multiple housekeeping control peptides, and can be applied to pre-treatment cancer tissue samples to predict the therapeutic effect of a targeted therapeutic agent.

### TECHNICAL FIELD

The present invention relates to a method of simultaneously quantifying various types of target proteins targeted by a targeting agent using nano-liquid chromatography (nano-LC)-triple quadrupole mass spectrometry.

### BACKGROUND ART

Most cancer-targeting agents target gene amplification, and predicting clinical response to these targeting agents is mainly based on how much of the relevant target protein is expressed, so information on whether and how much protein is overexpressed is the most helpful for clinicians in drug selection. Therefore, technology will be an innovative and useful invention if it may overcome the reality that it is difficult to quantify various proteins from clinical samples.

For FFPE biopsy, which is the most commonly obtained sample before targeted therapy in clinical practice, target genome analysis is relatively easy, but protein analysis at the level of global proteomic profiling is technically impossible by cross-linking due to the properties of the FFPE sample, and immunostaining is possible, but there is a limitation in throughput.

Accordingly, the present inventors, in order to develop technology for predicting the efficacy of a targeting agent in cancer patients before treatment, developed a method of simultaneously quantifying target peptides, which are predictive factors for efficacy of a targeting agent, in tryptic peptides isolated from pre-treatment cancer tissue using a nano-liquid chromatography-triple quadrupole system.

### DISCLOSURE

### Technical Problem

When the present invention is applied to a clinical situation in which both tumor tissue and adjacent normal tissue may be used, for example, endoscopic tissue of gastrointestinal cancer, the present invention is intended to provide a method of predicting response to a cancer-targeting agent, including (a) performing multiple reaction monitoring (MRM) mass spectrometry on target peptides and housekeeping control peptides in pre-treatment cancer tissue sample and its adjacent normal tissue sample isolated from a patient using triple quadrupole mass spectrometry and nano-liquid chromatography, (b) correcting the expression level of the target peptide measured in the multiple reaction monitoring (MRM) analysis to a relative expression level (ratio_{tumor/normal}) in tumor tissue to normal tissue with the housekeeping control peptide using Equation 1 and Equation 2 below:

$$amount \text{?} = AUC \text{?} / AUC \text{?} \times ( amount \text{?} / \text{?} total protein ) \qquad [ Equation 1 ]$$

$$Ratio \text{?} = \frac{amount \text{?}}{amount \text{?}} \times \text{?} [ \frac{amount \text{?}}{amount \text{?}} ] , \qquad [ Equation 2 ]$$

$$\text{?} \text{indicates text missing or illegible when filed}$$

and

(c) predicting clinical response to the targeting agent by determining the level of overexpression of the target peptide based on the corrected relative expression level (ratio_{tumor/normal}) result.

In addition, when the present invention is applied to a clinical situation in which only cancer tissue may be used and adjacent normal tissue cannot be used for biopsy, the present invention is intended to provide a method of predicting response to a targeted agent, including (a) performing multiple reaction monitoring (MRM) mass spectrometry on a target peptide and a housekeeping control peptide in a cancer tissue sample isolated from a patient using triple quadrupole mass spectrometry and nano-liquid chromatography, (b) obtaining a z_{normalized tissue amount} value by correcting the expression level of the target peptide measured in the multiple reaction monitoring (MRM) analysis with the housekeeping control peptide using Equation 1 and Equation 3 below and substituting the corrected value into a database,

[Equation 1]

[symbol]

[Equation 3]

[symbol]

(c) predicting clinical response to the targeting agent by determining the level of overexpression of the target peptide based on the z_{normalized tissue amount} result.

### Technical Solution

In order to solve the above problems, an aspect of the present invention provides a method of predicting response to a cancer-targeting agent, including (a) performing multiple reaction monitoring (MRM) mass spectrometry on target peptides and housekeeping control peptides in pre-treatment cancer tissue sample and its adjacent normal tissue sample isolated from a patient using triple quadrupole mass spectrometry and nano-liquid chromatography, (b) correcting the expression level of the target peptide measured in the multiple reaction monitoring (MRM) analysis to a relative expression level (ratio_{tumor/normal}) in tumor tissue to normal tissue with the housekeeping control peptide using Equation 1 and Equation 2 below:

$$amount \text{?} = AUC \text{?} / AUC \text{?} \times ( amount \text{?} / \text{?} total protein ) \qquad [ Equation 1 ]$$

$$Ratio \text{?} = \frac{amount \text{?}}{amount \text{?}} \times \text{?} [ \frac{amount \text{?}}{amount \text{?}} ] , \qquad [ Equation 2 ]$$

$$\text{?} \text{indicates text missing or illegible when filed}$$

and

(c) predicting clinical response to the targeting agent by determining the level of overexpression of the target peptide based on the corrected relative expression level (ratio_{tumor/normal}) result.

The sample may be isolated from a pre-treatment patient before immunotherapy.

The target peptide may include at least one selected from the group consisting of epidermal growth factor receptor (EGFR), MET proto-oncogene receptor tyrosine kinase (MET), fibroblast growth factor receptor 2 (FGFR2), erb-b2 receptor tyrosine kinase 2 (ERBB2), PD-Ll (CD274), TAP2 (TAP2), I23O1 (IDO1), SYWC (WARS1), and UB2L6 (UBE2L6), and the housekeeping control peptide may be mitogen-activated protein kinase 1 (MAPK1), cyclophilin B (PPIB), and beta-actin (β-actin, ACTB).

Here, EGFR may include the amino acid sequences of SEQ ID NO: 1 and SEQ ID NO: 2, MET may include the amino acid sequences of SEQ ID NO: 3 and SEQ ID NO: 4, FGFR2 may include the amino acid sequences of SEQ ID NO: 5 and SEQ ID NO: 6, ERBB2 may include the amino acid sequences of SEQ ID NO: 7 and SEQ ID NO: 8, PD-L1 (CD274) may include the amino acid sequences of SEQ ID NO: 9 and SEQ ID NO: 10, TAP2 (TAP2) may include the amino acid sequences of SEQ ID NO: 11 and SEQ ID NO: 12, I23O1 (IDO1) may include the amino acid sequences of SEQ ID NO: 13 and SEQ ID NO: 14, SYWC (WARS1) may include the amino acid sequence of SEQ ID NO: 15, and UB2L6 (UBE2L6) may include the amino acid sequence of SEQ ID NO: 16.

The target peptide may include EGFR, and when the relative expression level (ratio_{tumor/normal}) of EGFR is greater than 5, clinical response to the EGFR-targeting agent when used alone or in combination in the patient may be predicted to be high.

The target peptide may include MET, and when the relative expression level (ratio_{tumor/normal}) of MET is greater than 5, clinical response to the MET-targeting agent when used alone or in combination in the patient may be predicted to be high.

The target peptide may include FGFR2, and when the relative expression level (ratio_{tumor/normal}) of FGFR2 is greater than 5, clinical response to the FGFR2-targeting agent when used alone or in combination in the patient may be predicted to be high.

The target peptide may include ERBB2, and when the relative expression level (ratio_{tumor/normal}) of ERBB2 is greater than 5, clinical response to the HER2-targeting agent when used alone or in combination in the patient may be predicted to be high.

The target peptide may include i) PD-L1 (CD274) or ii) TAP2 (TAP2), I23O1 (IDO1), SYWC (WARS1), and UB2L6 (UBE2L6), and when the relative expression level (ratio_{tumor/normal}) of PD-L1 (CD274) is greater than 3 or quantification is possible only in tumor tissue, or when the average relative expression level (ratio_{tumor/normal}) of TAP2 (TAP2), I23O1 (IDO1), SYWC (WARS1), and UB2L6 (UBE2L6) is greater than 5, clinical response to the immunotherapy-targeting agent when used alone or in combination in the patient may be predicted to be high.

In the present invention, the cutoff of the ratio value, which is the criterion for selecting the targeting agent, is not limited to the above numerical value.

The cancer may be solid cancer.

The solid cancer may be gastrointestinal cancer.

Another aspect of the present invention provides a method of predicting response to a cancer-targeting agent, including (a) performing multiple reaction monitoring (MRM) mass spectrometry on a target peptide and a housekeeping control peptide in a gastric cancer tissue sample isolated from a patient using triple quadrupole mass spectrometry and nano-liquid chromatography, (b) obtaining a z_{normalized tissue amount} value by correcting the expression level of the target peptide measured in the multiple reaction monitoring (MRM) analysis with the housekeeping control peptide using Equation 1 and Equation 3 below and substituting the corrected value into a database,

[Equation 1]

[symbol]

[Equation 3]

[symbol]

(c) predicting clinical response to the targeting agent by determining the level of overexpression of the target peptide based on the z_{normalized tissue amount} result.

The target peptide may include at least one selected from the group consisting of epidermal growth factor receptor (EGFR), MET proto-oncogene receptor tyrosine kinase (MET), fibroblast growth factor receptor 2 (FGFR2), erb-b2 receptor tyrosine kinase 2 (ERBB2), PD-L1 (CD274), TAP2, I23O1 (IDO1), SYWC (WARS1), and UB2L6 (UBE2L6), and the housekeeping control peptide may be mitogen-activated protein kinase 1 (MAPK1), cyclophilin B (PPIB), and beta-actin (β-actin, ACTB).

The target peptide may include EGFR, and when the median z_{normalized tissue amount} value of EGFR is greater than 1.96, clinical response to the EGFR-targeting agent when used alone or in combination in the patient may be predicted to be high.

The target peptide may include MET, and when the median z_{normalized tissue amount} value of MET is greater than 1.96, clinical response to the MET-targeting agent when used alone or in combination in the patient may be predicted to be high.

The target peptide may include FGFR2, and when the median z_{normalized tissue amount} value of FGFR2 is greater than 1.96, clinical response to the FGFR2-targeting agent when used alone or in combination in the patient may be predicted to be high.

The target peptide may include ERBB2, and when the median z_{normalized tissue amount} value of ERBB2 is greater than 1.96, clinical response to the HER2-targeting agent when used alone or in combination in the patient may be predicted to be high.

The target peptide may include PD-L1 (CD274), and when the median z_{normalized tissue amount} value of PD-L1 (CD274) is greater than 1.96, clinical response to the immunotherapy (PD-L1/PD-1 antibody) when used alone or in combination in the patient may be predicted to be high (in the present invention, the cutoff of the z value, which is the criterion for selecting the targeting agent, is not limited to the above numerical value).

The normalized tissue amount in step (b) may be corrected with tumor purity using Equation 4, and the corrected value may be substituted into a purity-adjusted MRM database in gastric cancer to obtain z_{purity-adjusted amount} of each of PD-L1, ERBB2, EGFR, FGFR2, and MET peptides, and when the median value thereof is greater than 1.96, clinical response to the relevant targeting agent when used alone or in combination may be predicted to be high.

### Advantageous Effects

According to the present invention, a number of cancer-targeting molecules and therapeutic predictive factors can be quantified at the protein level through single analysis using triple quadrupole mass spectrometry-based multiple reaction monitoring (MRM) to provide clinicians with information about which of various targeting agents is expected to be the most effective.

The present invention is versatile because it can be directly applied to all types of solid cancer in which cancer tissue and normal tissue can be obtained in pairs, and exhibits high scalability of technology that can be used for target peptide quantification to predict the efficacy of new drugs by adding a target peptide in the future.

The amount of a protein sample required for single MRM analysis is about 150 μg, which can be sufficiently obtained from 2 pieces of endoscopic biopsy tissue, making it possible to obtain a sample with almost no delay in clinical procedures in most cases. Also, the present invention is relatively feasible to apply to actual clinical settings because each sample can be immediately analyzed, unlike existing proteomic analysis methods such as reverse phase protein array (RPPA) and the like, which require waiting until multiple samples are collected.

### DESCRIPTION OF DRAWINGS

FIGS. 1a to 1g show standard curves of light/heavy AUC ratio depending on the amount of each target peptide (light peptide), in which a shows standard curves of MAPK1_1, MAPK1_2, and PPIB, b shows standard curves of ACTB, EGFR_2, and EGFR_4, c shows standard curves of PDL1_1, PDL1_2, and ERBB2_4, d shows standard curves of ERBB2_5, FGFR2_1, and FGFR2_2, e shows standard curves of MET_2, MET_4, and I23O1_1, f shows standard curves of I23O1_2, TAP2_1, and TAP2_2, and g shows standard curves of UB2L6 and SYWC,

in which standard curves of three Q3 transitions used to identify each peptide are shown, and thereamong, the transition used for absolute quantification of the relevant peptide due to the largest AUC is marked with *;

FIG. 2 schematically shows a process of predicting efficacy of a targeting agent based on analysis results obtained by performing nano-LC-triple quadrupole multiple reaction monitoring (MRM) analysis on a pair of cancer tissue and normal tissue isolated from the same pre-treatment patient before immunotherapy at the same time point, upon application to gastrointestinal cancer, etc. in which both cancer tissue and normal tissue may be used for biopsy;

FIG. 3 schematically shows a process of predicting efficacy of a targeting agent based on analysis results obtained by performing nano-LC-triple quadrupole multiple reaction monitoring (MRM) analysis on cancer tissue alone, in a clinical situation using only cancer tissue isolated from a pre-treatment patient before immunotherapy without normal tissue;

FIG. 4 schematically shows an option that increases the precision of prediction by correcting the cancer tissue multiple reaction monitoring (MRM) quantitative value with tumor purity in a clinical situation using only cancer tissue isolated from a patient without normal tissue; and

FIGS. 5a to 5h show comparison of target peptide (light peptide) Q3 transition chromatograms of Examples, in which a to d show Skyline plots of Example 1, e shows Skyline plots of Example 2, f shows Skyline plots of Example 5, g shows Skyline plots of Example 8, and h shows Skyline plots of Example 10.

### BEST MODE

The present inventors simultaneously quantified a number of target peptides with housekeeping control peptides in tryptic peptides isolated from cancer tissue samples using nano-liquid chromatography-triple quadrupole, and also developed an algorithm for predicting response to a targeting agent in a patient in clinical practice based on results of determining the level of overexpression, and ascertained that actual clinical response to the targeting agent is consistent with results of the algorithm of the present invention, thus culminating in the present invention.

For cases in which both pre-treatment tumor and normal tissues may be used, clinical response is predicted using the relative quantitative value (ratio) of the peptide in tumor tissue compared to normal tissue. Specifically, the present invention pertains to a method of predicting response to a targeting agent, including (a) performing multiple reaction monitoring (MRM) mass spectrometry on target peptides and housekeeping control peptides in pre-treatment cancer tissue sample and its adjacent normal tissue sample isolated from a patient using triple quadrupole mass spectrometry and nano-liquid chromatography, (b) correcting the expression level of the target peptide measured in the multiple reaction monitoring (MRM) analysis to a relative expression level (ratiotumor/normal) in tumor tissue to normal tissue with the housekeeping control peptide using Equation 1 and Equation 2 below:

$$amount \text{?} = AUC \text{?} / AUC \text{?} \times ( amount \text{?} / \text{?} total protein ) \qquad [ Equation 1 ]$$

$$Ratio \text{?} = \frac{amount \text{?}}{amount \text{?}} \times \text{?} [ \frac{amount \text{?}}{amount \text{?}} ] , \qquad [ Equation 2 ]$$

$$\text{?} \text{indicates text missing or illegible when filed}$$

and

(c) predicting clinical response to the targeting agent by determining the level of overexpression of the target peptide based on the corrected relative expression level (ratio_{tumor/normal}) result.

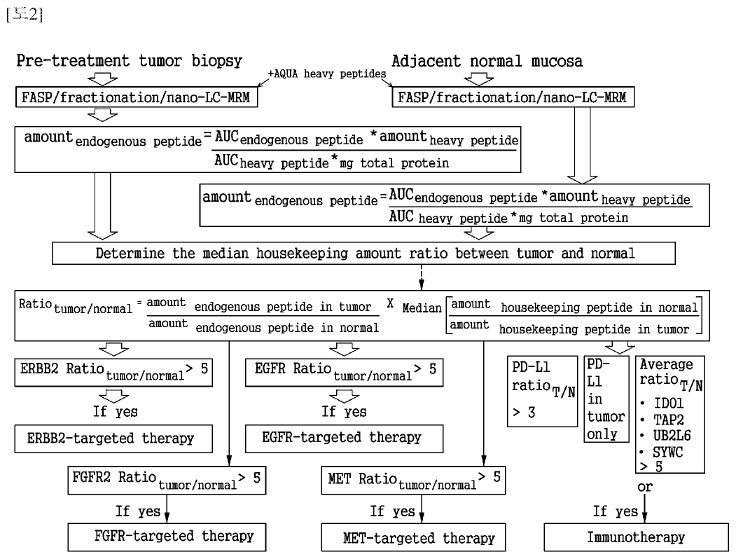

FIG. 2 schematically shows a process of predicting response to a cancer-targeting agent by performing multiple reaction monitoring (MRM) mass spectrometry on pre-treatment cancer tissue and normal tissue samples according to an embodiment of the present invention.

With reference to FIG. 2, triple quadrupole multiple reaction monitoring is performed on respective proteins of a cancer tissue sample and a normal tissue sample isolated from the same patient using absolute protein quantitation (AQUA) peptides, and the expression level of the target peptide measured in the MRM analysis is corrected to a relative expression level (ratio_{tumor/normal}) in tumor tissue to normal tissue with the housekeeping control peptide using Equation 1 and Equation 2, and when the relative expression level (ratio_{tumor/normal}) of each target peptide exceeds a predetermined value, clinical efficacy of the targeting agent corresponding to each target peptide is predicted to be high.

In an embodiment of the present invention, proteins were isolated from a cancer tissue sample and a normal tissue sample isolated from the same patient, and then peptidized by performing FASP (filter aided sample preparation) reaction overnight, followed by analysis with nano-chromatography-triple quadrupole using chip LC together with AQUA peptides in Table 1 (target peptide) and Table 2 (housekeeping control peptide), thereby obtaining information on all protein expression amounts necessary for predicting the efficacy of the targeting agent in cancer through single analysis.

The present invention is not limited to chip LC and may be applied to any nano-LC column.

The sample may be a frozen sample or an RNA later sample.

The sample may be isolated from a pre-treatment patient before immunotherapy.

In the present invention, the cancer tissue sample and the normal tissue sample may be isolated from the same pre-treatment patient before immunotherapy at the same time point.

The target peptide of the present invention may include at least one selected from the group consisting of epidermal growth factor receptor (EGFR), MET proto-oncogene receptor tyrosine kinase (MET), fibroblast growth factor receptor 2 (FGFR2), erb-b2 receptor tyrosine kinase 2 (ERBB2), PD-L1 (CD274), TAP2 (TAP2), I23O1 (IDO1), SYWC (WARS1), and UB2L6 (UBE2L6), and the housekeeping control peptide may be mitogen-activated protein kinase 1 (MAPK1), cyclophilin B (PPIB), and beta-actin (β-actin, ACTB).

Here, EGFR may include the amino acid sequences of SEQ ID NO: 1 and SEQ ID NO: 2, MET may include the amino acid sequences of SEQ ID NO: 3 and SEQ ID NO: 4, FGFR2 may include the amino acid sequences of SEQ ID NO: 5 and SEQ ID NO: 6, ERBB2 may include the amino acid sequences of SEQ ID NO: 7 and SEQ ID NO: 8, PD-L1 (CD274) may include the amino acid sequences of SEQ ID NO: 9 and SEQ ID NO: 10, TAP2 (TAP2) may include the amino acid sequences of SEQ ID NO: 11 and SEQ ID NO: 12, I23O1 (IDO1) may include the amino acid sequences of SEQ ID NO: 13 and SEQ ID NO: 14, SYWC (WARS1) may include the amino acid sequence of SEQ ID NO: 15, and UB2L6 (UBE2L6) may include the amino acid sequence of SEQ ID NO: 16.

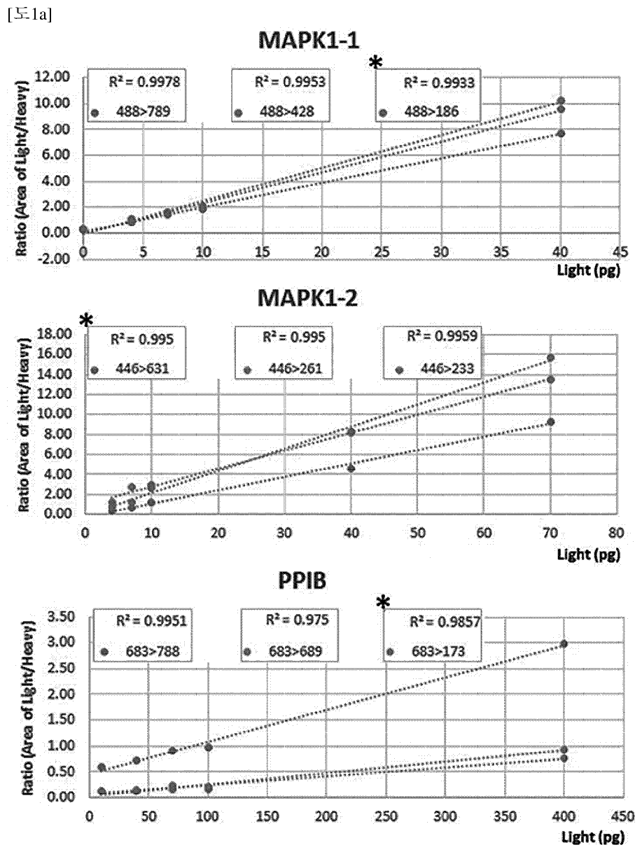

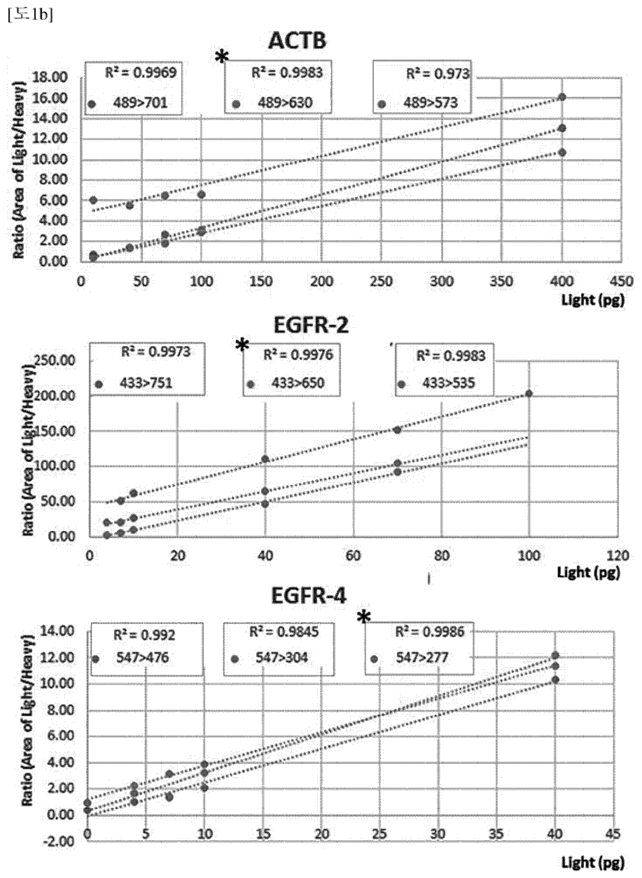

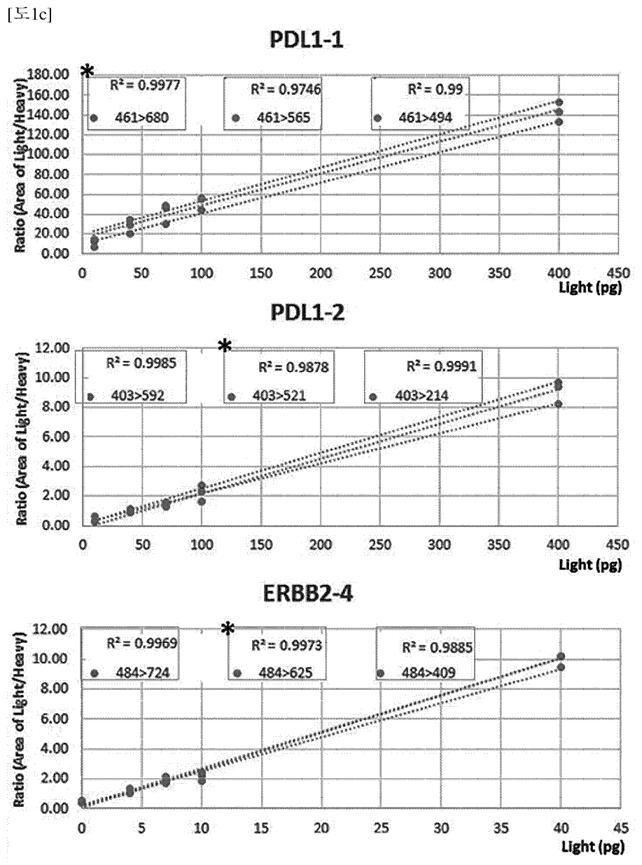

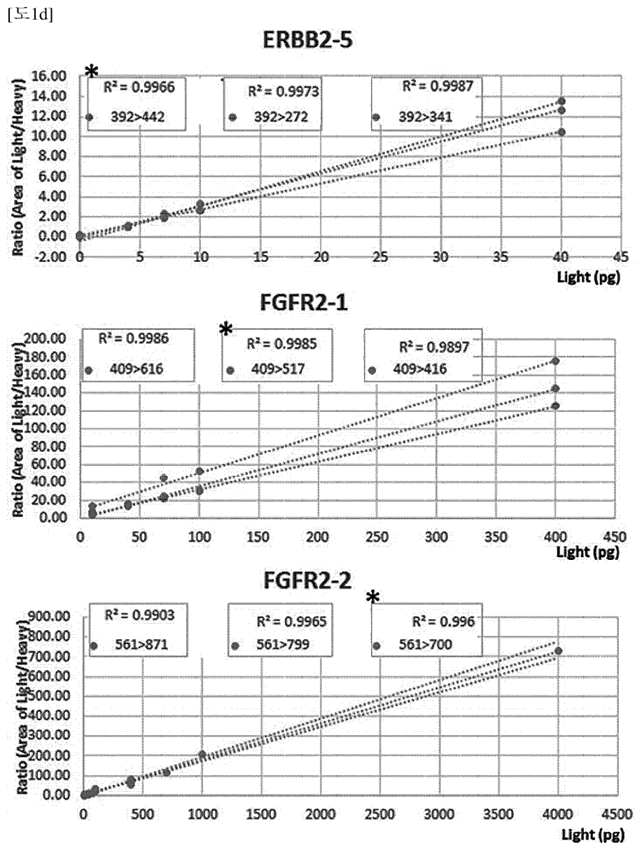

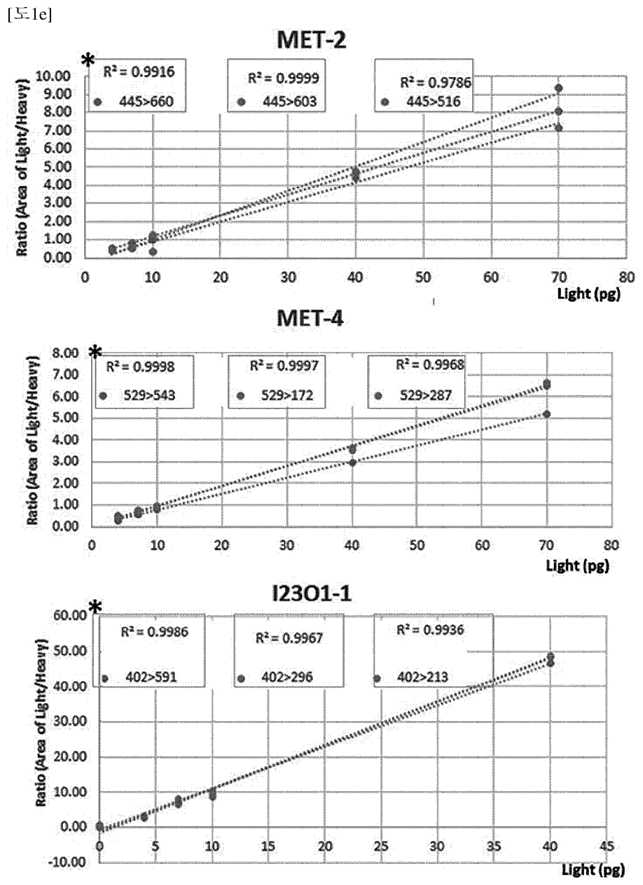

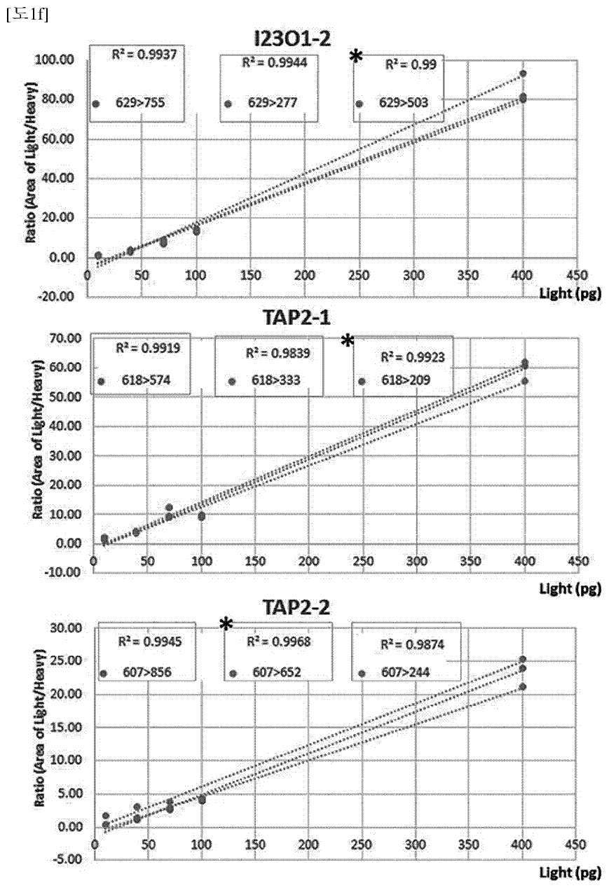

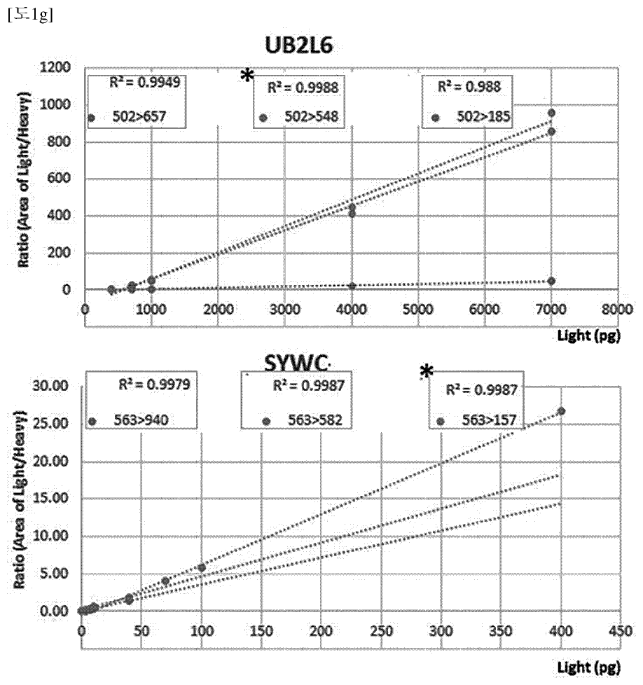

MRM raw data for EGFR, FGFR2, ERBB2, MET, IDO1, WARS1, TAP2, UB2L6, GBP1, CD274 and housekeeping control peptides co-quantified in Examples of the present invention were transferred to the Skyline program (software), and the chromatographic peak AUC of Q3 transition was absolutely quantified with a standard curve. For peptides identified in multiple fractions, Q3 transition AUCs of individual fractions were summed. Standard curves were obtained by performing MRM on a certain amount of AQUA in an E. coli protein (tryptic digest of E. coli lysate) and on various proportions of light endogenous synthetic peptide (FIG. 1).

In order to accurately and reproducibly generate mass spectrometric data for ultimate clinical use, raw data obtained by single MRM mass spectrometry for each of tumor tissue and normal tissue was normalized with a housekeeping control peptide to afford an expression ratio of tumor tissue to normal tissue, from which the protein quantitative value was calculated using Equation 1 and Equation 2.

Specifically, as in Equation 1, the amount of each peptide (endogenous peptide; light peptide) based on the spike-in AQUA peptide (heavy peptide) was obtained using a standard curve.

[Equation 1]

[symbol]

Then, as in Equation 2, the amount of the target peptide (endogenous peptide; light peptide) in Table 1 was normalized by being divided by the amount of the housekeeping control peptide, after which the relative expression level was represented as a ratio between the relevant tumor and normal (ratio_{tumor/normal}).

$$Ratio \text{?} = \frac{amount \text{?}}{amount \text{?}} \times \text{?} [ \frac{amount \text{?}}{amount \text{?}} ] , \qquad [ Equation 2 ]$$

$$\text{?} \text{indicates text missing or illegible when filed}$$

For PD-L1, EGFR, ERBB2, FGFR2, and MET, one of two peptide MRM values assigned to each protein based on the indicator set by the analyst (e.g. average AUC) may be taken as a representative value of the relevant protein.

Based on results of ratio_{tumor/normal}, it is possible to predict whether the target peptide (target protein) is overexpressed and clinical efficacy of the relevant targeting agent.

The higher the expression level of the target peptide (target protein), the higher the drug response. Therefore, the cutoff of the ratio, which is the criterion for selecting the targeting agent, is not limited to the following numerical value.

In an embodiment of the present invention, as shown in Table 5, when the ratio_{tumor/normal} of the target peptides of EGFR, MET, FGFR2, and ERBB2 was greater than 5, clinical response to the relevant targeting agent (EGFR-targeting agent, MET-targeting agent, FGFR2-targeting agent, HER2-targeting agent) when used alone or in combination was predicted to be high.

Also, when the relative expression level (ratio_{tumor/normal}) of PD-L1 (CD274) was greater than 3 or quantification was possible only in tumor tissue, or when the average relative expression level (ratio_{tumor/normal}) of six peptides representing TAP2 (TAP2), I23O1 (IDO1), SYWC (WARS1), and UB2L6 (UBE2L6) was greater than 5, clinical response to the immunotherapy-targeting agent when used alone or in combination in the patient was predicted to be high.

Therefore, in the present invention, the target peptide may include EGFR, and when the relative expression level (ratio_{tumor/normal}) of EGFR is greater than 5, clinical response to the EGFR-targeting agent when used alone or in combination in the patient may be predicted to be high.

Also, the target peptide may include MET, and when the relative expression level (ratio_{tumor/normal}) of MET is greater than 5, clinical response to the MET-targeting agent when used alone or in combination in the patient may be predicted to be high.

Also, the target peptide may include FGFR2, and when the relative expression level (ratio_{tumor/normal}) of FGFR2 is greater than 5, clinical response to the FGFR2-targeting agent when used alone or in combination in the patient may be predicted to be high.

Also, the target peptide may include ERBB2, and when the relative expression level (ratio_{tumor/normal}) of ERBB2 is greater than 5, clinical response to the HER2-targeting agent when used alone or in combination in the patient may be predicted to be high.

Also, the target peptide may include PD-L1 (CD274) or TAP2 (TAP2), I23O1 (IDO1), SYWC (WARS1), and UB2L6 (UBE2L6) peptides, and when the relative expression level (ratio_{tumor/normal}) of PD-L1 (CD274) is greater than 3 or quantification is possible only in tumor tissue, or when the average relative expression level (ratio_{tumor/normal}) of TAP2 (TAP2), I23O1 (IDO1), SYWC (WARS1), and UB2L6 (UBE2L6) immunoproteins (i.e. average value of T/N ratio_{I23O1_1}, T/N ratio_{I23O1_2}, T/N ratio_{TAP2_1}, T/N ratio_{TAP2_2}, T/N ratio_{SYWC}, and T/N ratio_{UB2L6}) is greater than 5, clinical response to the immunotherapy-targeting agent when used alone or in combination in the patient may be predicted to be high.

In the present invention, the average relative expression level (ratio_{tumor/normal}) of TAP2 (TAP2), I23O1 (IDO1), SYWC (WARS1), and UB2L6 (UBE2L6) may be the average value of relative expression levels of six peptides representing TAP2 (TAP2), I23O1 (IDO1), SYWC (WARS1), and UB2L6 (UBE2L6) (TAP2 (TAP2) peptides including the amino acid sequences of SEQ ID NO: 11 and SEQ ID NO: 12, I23O1 (IDO1) peptides including the amino acid sequences of SEQ ID NO: 13 and SEQ ID NO: 14, SYWC (WARS1) peptide including the amino acid sequence of SEQ ID NO: 15, and UB2L6 (UBE2L6) peptide including the amino acid sequence of SEQ ID NO: 16).

In order to verify the accuracy of the prediction method in Examples of the present invention, based on results of analyzing patient 1 who had clinical response to immunotherapy, the average value of T/N ratio_{I23O1_1}, T/N ratio_{I23O1_2}, T/N ratio_{TAP2_1}, T/N ratio_{TAP2_2}, T/N ratio_{SYWC}, and T/N ratio_{UB2L6} was confirmed to be 9.7 (Table 7).

In contrast, for gastric cancer patients 2 and 3 who had no clinical response to immunotherapy, the average values of T/N ratio_{I23O1_1}, T/N ratio_{I23O1_2}, T/N ratio_{TAP2_1}, T/N ratio ratio_{TAP2_2}, T/N ratio_{SYWC}, and T/N ratio_{UB2L6} were 2.2 and 2.6, respectively (Table 7), and the PD-L1 value was 0, indicating that neither of two conditions required to be predicted to respond to immunotherapy according to the present invention was satisfied.

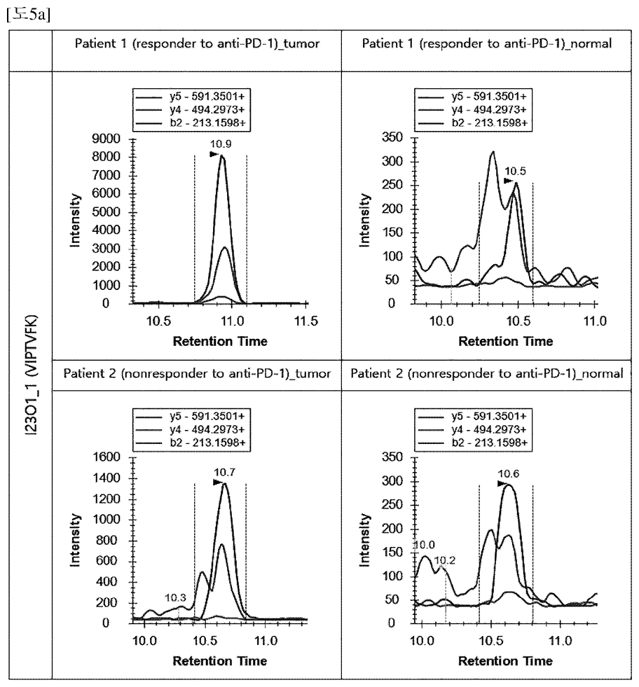

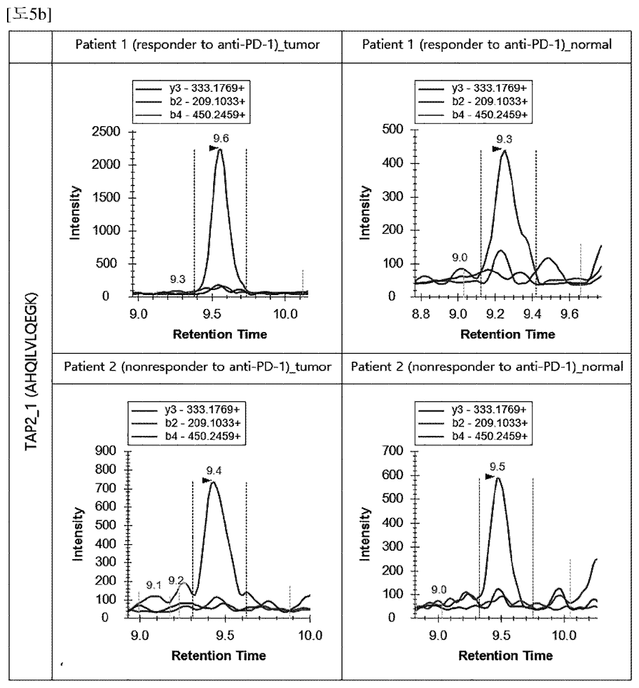

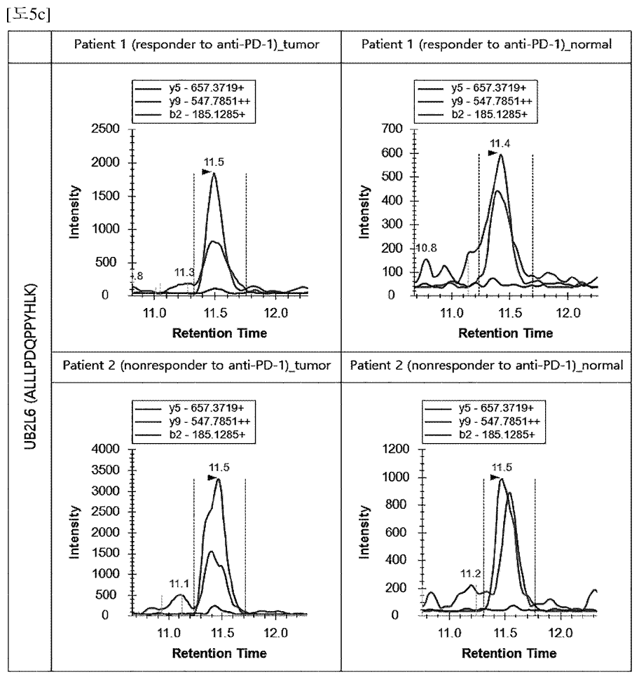

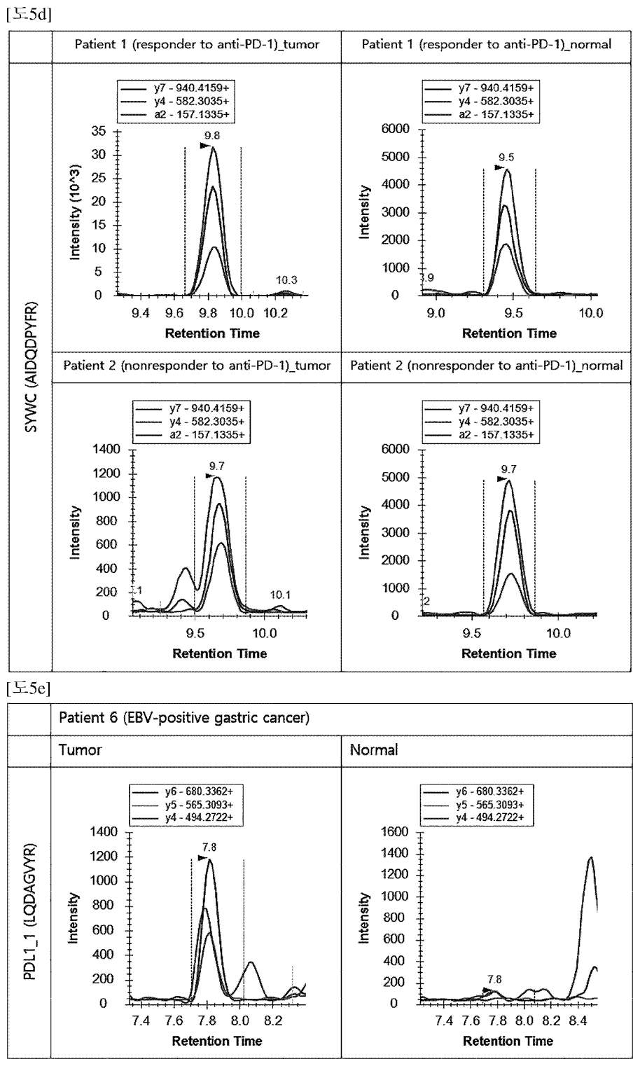

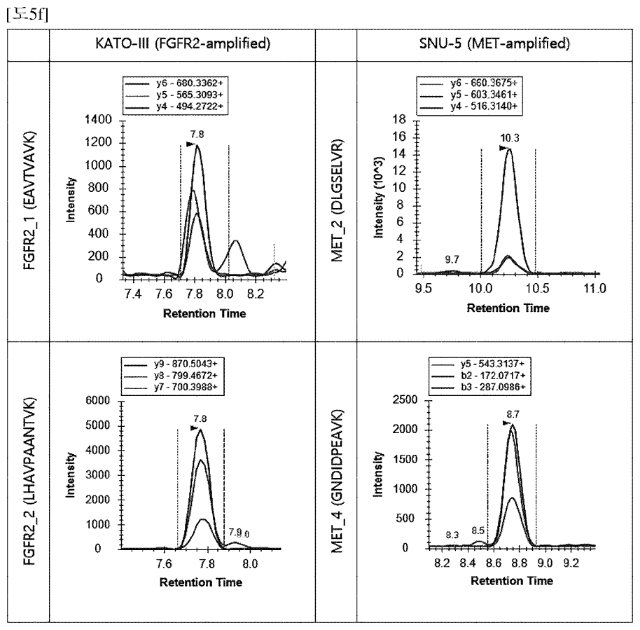

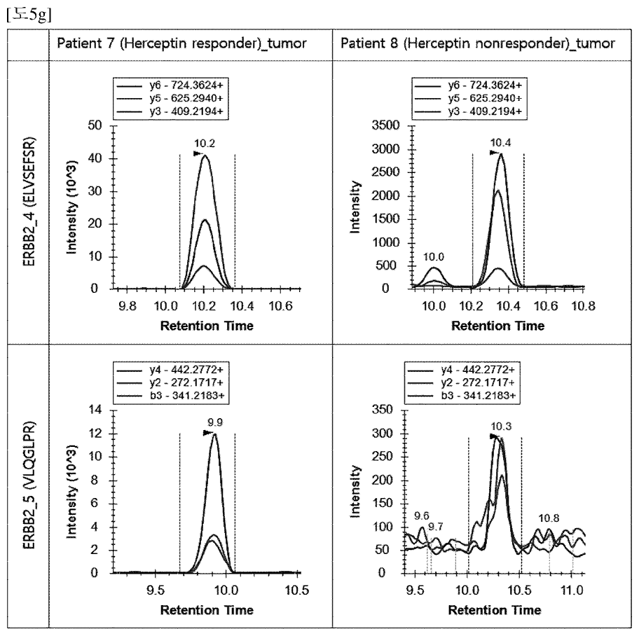

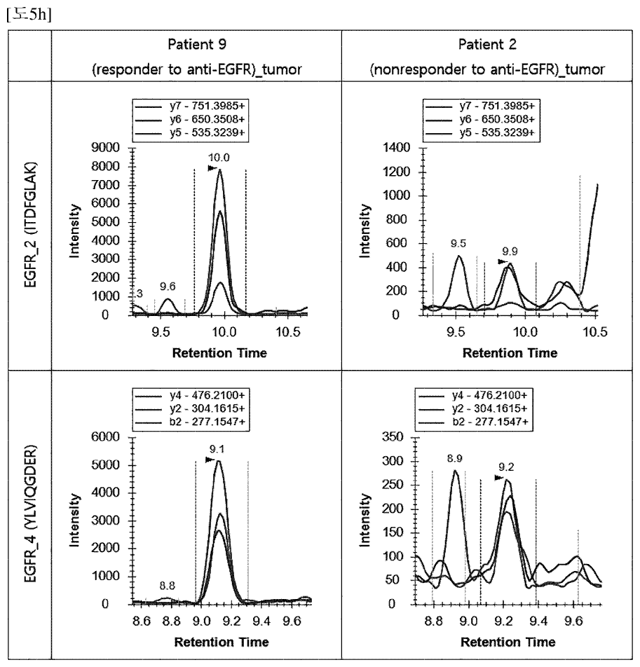

FIGS. 5a to 5d show representative chromatograms of tumor and normal of patients 1 and 2.

As such, it was confirmed that the prediction method according to the present invention was consistent with results of actual clinical response to the immunotherapy.

Based on results of verifying the prediction of clinical response to the EGFR-targeting agent in a male gastric cancer patient (patient 9) with overexpression of EGFR protein on immunostaining due to strong EGFR 3+(range, 0˜3+) positive on EGFR immunostaining and a female gastric cancer patient (patient 2) with EGFR negative on immunostaining, the median value of the housekeeping control-normalized tumor/normal EGFR ratio in patient 9 who had clinical response to EGFR antibody therapy was 159.1 or 13.6, indicating the results of prediction in which targeted therapy was recommended due to EGFR protein overexpression (EGFR ratio_{tumor/normal}>5)

On the other hand, the median value of the housekeeping control-normalized EGFR tumor/normal ratio in patient 2 who had no clinical response to the same EGFR antibody therapy was 1 or 1, indicating ratio_{tumor/normal}<5. The above results show the clinical validity of the present invention for predicting clinical response to the EGFR-targeting agent. FIG. 5h shows representative chromatograms of tumors of patient 9 and patient 2 for comparison.

The cancer may be gastrointestinal cancer in which tumor tissue and adjacent normal tissue may be easily used for biopsy, and the present invention may be applied to any solid cancer in which normal tissue and tumor tissue may be obtained in pairs at the same time point/organ (tissue type) before treatment.

The present invention may also be applied to prediction of clinical response not only to therapy for metastatic cancer regardless of the line of therapy but also to adjuvant or neoadjuvant therapy.

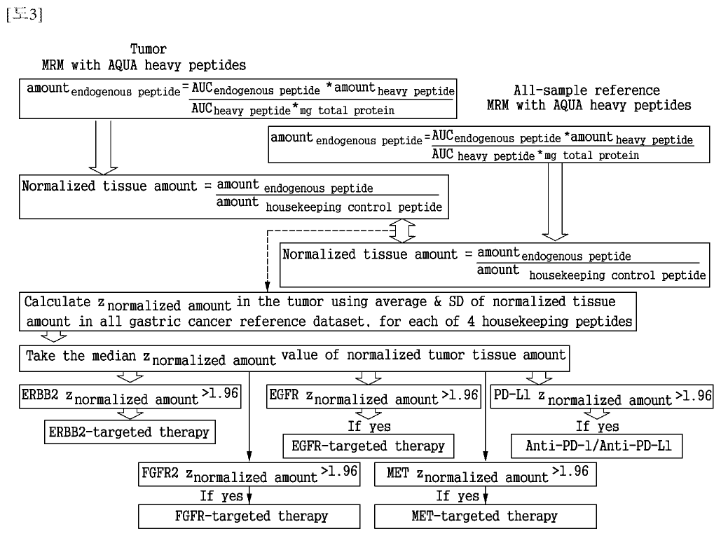

Depending on the clinical situation, there may be cases where only tumor tissue is available for MRM analysis and adjacent normal tissue is not available. As such, as shown in FIG. 3, after normalization with a housekeeping control peptide, the level of protein overexpression in the tumor sample may be quantified by MRM.

In this application, the present invention pertains to a method of predicting response to a targeting agent, including (a) performing multiple reaction monitoring (MRM) mass spectrometry on a target peptide and a housekeeping control peptide in a cancer tissue sample isolated from a patient using triple quadrupole mass spectrometry and nano-liquid chromatography, (b) obtaining a z_{normalized tissue amount} value by correcting the expression level of the target peptide measured in the multiple reaction monitoring (MRM) analysis with the housekeeping control peptide using Equation 1 and Equation 3 below and substituting the corrected value into a database,

[Equation 1]

[symbol]

[Equation 3]

[symbol] , and

(c) predicting clinical response to the targeting agent by determining the level of overexpression of the target peptide based on the z_{normalized tissue amount} result.

FIG. 3 shows a process of predicting response to a cancer-targeting agent by performing multiple reaction monitoring (MRM) mass spectrometry only on a pre-treatment cancer tissue sample according to an embodiment of the present invention.

In the present invention, as in Equation 3, the amount of the target peptide (endogenous peptide; light peptide) may be normalized by being divided by the amount of the housekeeping control peptide.

[Equation 3]

[symbol]

How high the normalized tissue amount of each tumor sample is compared to the average normalized tissue amount of all-tumor MRM database is determined.

Determining the level of overexpression of the target peptide is to evaluate how high the normalized tissue amount of each tumor sample corrected above is compared to the average normalized tissue amount of all-tumor MRM database in the relevant tumor (tissue type).

The all-tumor MRM database is composed of values calculated by applying the present invention to the same protocol for each tissue type. Here, the median z value obtained by normalization with the housekeeping control peptide is used as a representative value for final determination.

Specifically, in an embodiment of the present invention, when the normalized tissue amount showed a high value exceeding the standard deviation of +1.96 from the average normalized amount of all-tumor MRM database (5% outlier), the relevant peptide was evaluated to be overexpressed, based on which the efficacy of the relevant agent was predicted to be high.

The target peptide may include at least one selected from the group consisting of epidermal growth factor receptor (EGFR), MET proto-oncogene receptor tyrosine kinase (MET), fibroblast growth factor receptor 2 (FGFR2), erb-b2 receptor tyrosine kinase 2 (ERBB2), PD-L1 (CD274), TAP2 (TAP2), I23O1 (IDO1), SYWC (WARS1), and UB2L6 (UBE2L6), and the housekeeping control peptide may be mitogen-activated protein kinase 1 (MAPK1), cyclophilin B (PPIB), and beta-actin (β-actin, ACTB).

The target peptide may include EGFR, and when the median z_{normalized tissue amount} value of EGFR is greater than 1.96, clinical response to the EGFR-targeting agent when used alone or in combination in the patient may be predicted to be high.

The target peptide may include MET, and when the median z_{normalized tissue amount} value of MET is greater than 1.96, clinical response to the MET-targeting agent when used alone or in combination in the patient may be predicted to be high.

The target peptide may include FGFR2, and when the median z_{normalized tissue amount} value of FGFR2 is greater than 1.96, clinical response to the FGFR2-targeting agent when used alone or in combination in the patient may be predicted to be high.

The target peptide may include ERBB2, and when the median z_{normalized tissue amount} value of ERBB2 is greater than 1.96, clinical response to the HER2-targeting agent when used alone or in combination in the patient may be predicted to be high.

The target peptide may include PD-L1 (CD274), and when the median z_{normalized tissue amount} value of PD-L1 (CD274) is greater than 1.96, clinical response to the immunotherapy (PD-L1/PD-1 antibody) when used alone or in combination in the patient may be predicted to be high (a representative chromatogram of PD-L1 is shown in FIG. 5e).

In Examples of the present invention, the median value of normalized tissue ERBB2 amount in patient 7 who had clinical response to ERBB2 antibody therapy was z=76.0 or z=67.9, based on which the inventive results predicted to have high clinical efficacy of the ERBB2-targeting agent (z_{normalized tissue amount}>1.96) were obtained, whereas the median value of normalized tissue ERBB2 amount in gastric cancer of patient 8 who had no clinical response to the same therapy was z=0.43 or z=0.84, which was predicted to have low clinical efficacy of the ERBB2-targeting agent (z_{normalized tissue amount}<1.96). Both of the above two cases were determined to be HER2-positive gastric cancer based on pathological examination results and received Herceptin (trastuzumab) administration, but only patient 7 was judged to be expected to be efficacious based on the results of the present invention. Therefore, the present invention exhibited superior results consistent with actual clinical response compared to typical pathological tests, thus demonstrating the clinical validity of the present invention for predicting clinical response to the targeting agent. FIG. 5g shows representative chromatograms of tumors of patients 7 and 8 for comparison.

In addition, MRM was performed by the method of FIG. 3 in two gastric cancer cell lines having a significant difference in response depending on the type of immunotherapy, and the prediction result of response to the targeting agent was verified. IC₅₀ of rogaratinib as an FGFR inhibitor was confirmed to be 30.977 nM in a KATO-III cell line, which is an FGFR2-amplified cell line, but exceeded 40 nM (>40 nM) in SNU5 cells, confirming low response to the FGFR inhibitor. In contrast, IC₅₀ of crizotinib as a MET inhibitor was 22.61 nM in a KATO-III cell line, but was 17.53 nM in SNU-5, which is a MET-amplified cell line, confirming that response to the MET inhibitor was significantly higher in SNU-5 than in KATO-III. Based on results of MRM, the median z_{normalized tissue amount} value of housekeeping control-normalized MET was z=−0.19 or z=0.58 in KATO-III, but was z=2.77 or z=2.26 in SNU-5, so that response to the MET inhibitor was predicted to be high in SNU-5 with z_{normalized tissue amount}>1.96.

This coincided with experimental data of the inventors in which IC₅₀ of crizotinib as the MET inhibitor was relatively low in SNU-5 and thus response to the MET inhibitor was relatively high.

The median z_{normalized tissue amount} value of housekeeping control-normalized FGFR2 was z=−0.43 or z=−0.32 in SNU-5, but was z=2.25 or z=2.85 in KATO-III, so that response to the FGFR inhibitor in KATO-III with z_{normalized tissue amount}>1.96 was predicted to be high. This coincided with experimental data of the inventors in which IC₅₀ of rogaratinib as the FGFR inhibitor was relatively low in KATO-III and thus response to the FGFR inhibitor was relatively high (FIG. 5f).

Also, based on results of verifying the prediction effect of clinical response to the EGFR-targeting agent by the method of FIG. 3 using only the cancer tissue sample isolated from a pre-treatment patient before immunotherapy, the median value of normalized tissue EGFR amount in patient 9 who had clinical response to EGFR antibody therapy was z=2.38 or z=2.67, whereby this gastric cancer tissue was predicted to have high clinical efficacy of the EGFR-targeting agent due to EGFR protein overexpression (z_{normalized tissue amount}>1.96). However, the median value of normalized tissue EGFR amount in gastric cancer of patient 2 who had no clinical response to the same therapy was z=−0.57 or z=−0.37, which was predicted to have low clinical efficacy of the EGFR-targeting agent (z_{normalized tissue amount}<1.96).

Thus, it can be found that the prediction method of the present invention according to FIG. 3 is also valid for predicting clinical response to the EGFR-targeting agent.

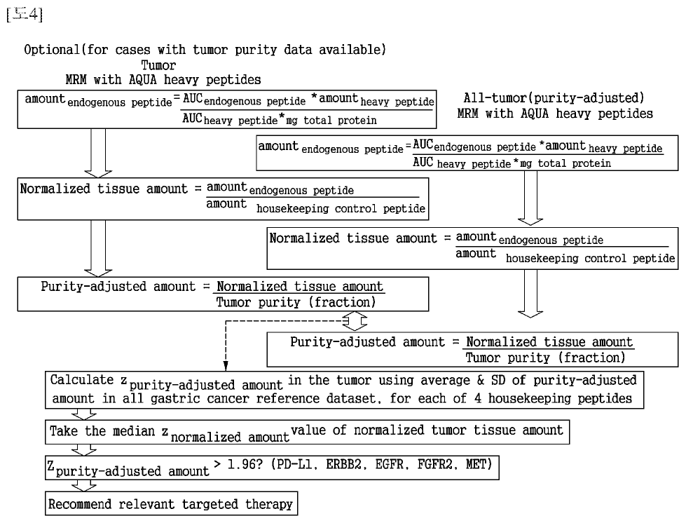

FIG. 4 schematically shows an option that increases the precision of prediction by correcting the cancer tissue multiple reaction monitoring (MRM) quantitative value with tumor purity, in a clinical situation using only cancer tissue isolated from a pre-treatment patient before immunotherapy without normal tissue, according to the present invention.

In the present invention, the normalized tissue amount in step (b) may be corrected with tumor purity using Equation 4, and the corrected value may be substituted into a purity-adjusted MRM database in gastric cancer to obtain z_{purity-adjusted amount} of each of PD-L1, ERBB2, EGFR, FGFR2, and MET, and when the median value thereof exceeds 1.96, clinical response to the relevant targeting agent when used alone or in combination may be predicted to be high.

[Equation 4]

[symbol]

When evaluating how high the normalized tissue amount of each tumor sample is compared to the average normalized tissue amount of all-sample MRM database, in order to increase the precision of prediction, the all-sample MRM database may be composed of MRM data corrected with tumor purity, and the invention may be optionally applied by correcting the MRM value with the tumor purity value calculated by a method such as NGS, etc. For human cancer biopsy tissue, especially gastric cancer biopsy, this correction serves to correct the bias in which the overall low and highly variable tumor purity affects measurement of the target peptide expression level, which reflects the knowledge of cancer biology in which the amount of the relevant target peptide is higher in tumor tissue than in normal tissue.

To this end, the normalized tissue amount calculated by Equation 3 may be divided by the tumor purity as the denominator determined using algorithms such as ABSOLUTE, Sequenza, PureCN, etc. after whole genome sequencing (WGS) or targeted DNA sequencing of genomic DNA isolated from the same tissue as the tumor tissue subjected to MRM, thereby obtaining the purity-adjusted tissue amount of PD-L1, ERBB2, EGFR, FGFR2, or MET (Equation 4).

In an embodiment of the present invention, when the median z_{purity-adjusted amount} value obtained by normalization with a housekeeping control peptide using Equation 4 exceeded the standard deviation of +1.96 from the average purity-adjusted amount of all-tumor MRM database (z_{purity-adjusted amount}>1.96; 5% outlier), the efficacy of the relevant targeting agent was predicted to be high, and based on results of verification thereof, the median z_{purity-adjusted amount} value in gastric cancer of patient 9 who had clinical response to the EGFR-targeting agent was 2.64 or 2.65, indicating the inventive results predicted to have high clinical efficacy (z_{purity-adjusted amount}>1.96), whereas the median z_{purity-adjusted amount} value in gastric cancer of patient 2 who had no clinical response to the same therapy was −0.44 or −0.44, which was predicted to have low clinical efficacy (z_{purity-adjusted amount}<1.96), ultimately demonstrating the clinical validity of the present invention for predicting clinical response.

### Mode for Invention

A better understanding of the configuration and effects of the present invention may be obtained through the following examples. These examples are merely set forth to illustrate the present invention, and are not to be construed as limiting the scope of the present invention.

### <Materials and Experimental Methods>

### 1. Preparation of AQUA (Absolute Protein Quantitation) Peptide

A certain amount of custom-made AQUA (absolute protein quantitation) peptide (Tables 1 and 2) was administered (“spiked in”), followed by quantification using a standard curve of the chromatographic peak AUC of unique transition of a target peptide (endogenous peptide; light peptide) contained in each tumor or normal sample based thereon.

Thereafter, the quantitative value of each target peptide was normalized with the quantitative value of a housekeeping control peptide obtained in the same manner.

The AQUA peptide used was an AQUA peptide in which one amino acid was substituted with heavy isotope (¹³C/¹⁵N). Since AQUA and a target peptide (endogenous peptide; light peptide) have the same chromatographic chemical properties in Q₁, they may be specifically distinguished and quantified based on different information of Q₃. Triple quadrupole mass spectrometry is a method of quantifying tryptic peptide with a combination of a precursor ion (Q₁) up to 1,400 m/z and a product ion (Q₃). Q₁ and Q₃ are connected in tandem as independent quadrupole mass filters to quantify the precursor ion and the product ion, respectively.

Various candidate peptide sequences for each target protein to be analyzed were made into AQUA peptides, and mass spectrometry was performed, followed by optimization with a peptide having the highest selectivity thereamong.

An AQUA peptide was combined with a peptide sequence having high nano-liquid chromatography-triple quadrupole selectivity while avoiding the overlap of chromatographic properties in order to obtain information on all protein expression amounts necessary for predicting the efficacy of a targeting agent in cancer through single analysis. (Table 1 and Table 2).

Table 1 below shows retention time (RT) and transition of the stable isotope AQUA peptide.

(“*” represents ¹³C/¹⁵N stable isotope labeling.)

TABLE 1

| | Dose | Amino acid | Retention | | | |
|---|---|---|---|---|---|---|
| Uniprot | (ng) | sequence | time (RT) | | Q₁ > Q₃ (CE) | |
| EGFR_2(P00533) | 2.5 | ITDFGLA*K | 10 | (±0.3) | 434.7 > 755.4 | (14.4) |
| | | (SEQ ID NO: 1) | | | 434.7 > 654.4 | (11.4) |
| | | | | | 434.7 > 539.3 | (17.4) |
| EGFR_4(P00533) | 12.5 | YLVIQGDE*R | 9.3 | (±0.4) | 549.8 > 482.2 | (13) |
| | | (SEQ ID NO: 2) | | | 549.8 > 310.2 | (23) |
| | | | | | 549.8 > 277.2 | (23) |
| MET_2(P08581) | 25 | DLGSELV*R | 10.4 | (±0.4) | 447.7 > 666.4 | (14.8) |
| | | (SEQ ID NO: 3) | | | 447.7 > 609.4 | (14.8) |
| | | | | | 447.7 > 522.3 | (11.8) |
| MET_4(P08581) | 50 | GNDIDPEAV*K | 8.5 | (±0.2) | 532.3 > 549.3 | (22) |
| | | (SEQ ID NO: 4) | | | 532.3 > 172.1 | (17) |
| | | | | | 532.3 > 287.1 | (12) |
| FGFR2_1(P21802) | 12.5 | EAVTVAV*K | 8.3 | (±0.5) | 411.8 > 622.4 | (10.7) |
| | | (SEQ ID NO: 5) | | | 411.8 > 523.3 | (13.7) |
| | | | | | 411.8 > 422.3 | (10.7) |
| FGFR2_2(P21802) | 25 | LHAVPAANTV*K | 7.8 | (±0.4) | 563.8 > 876.5 | (15) |
| | | (SEQ ID NO: 6) | | | 563.8 > 805.5 | (18.4) |
| | | | | | 563.8 > 706.4 | (21.4) |
| ERBB2_4(P04626) | 12.5 | ELVSEFS*R | 9.6 | (±0.4) | 485.8 > 728.4 | (11) |
| | | (SEQ ID NO: 7) | | | 485.8 > 629.3 | (11) |
| | | | | | 485.8 > 413.2 | (21) |
| ERBB2_5(P04626) | 12.5 | VLQGL*PR | 8.8 | (±0.3) | 395.3 > 449.3 | (18) |
| | | (SEQ ID NO: 8) | | | 395.3 > 272.2 | (8) |
| | | | | | 395.3 > 341.2 | (8) |
| PDL1_1(Q9NZQ7) | 12.5 | LQDAGVYR* | 8 | (±0.4) | 466.2 > 690.3 | (15.3) |
| | | (SEQ ID NO: 9) | | | 466.2 > 575.3 | (18.3) |
| | | | | | 466.2 > 504.3 | (15.3) |
| PDL1_2(Q9NZQ7) | 250 | VNAPYNK* | 7 | (±0.2) | 407.2 > 600.3 | (10.5) |
| | | (SEQ ID NO: 10) | | | 407.2 > 529.3 | (13.5) |
| | | | | | 407.2 > 432.2 | (13.5) |
| TAP2_1(Q03519) | 25 | AHQILVLQEG*K | 9.4 | (±0.3) | 619.9 > 336.2 | (25) |
| | | (SEQ ID NO: 11) | | | 619.9 > 209.1 | (15) |
| | | | | | 619.9 > 450.2 | (25) |
| TAP2_2(Q03519) | 50 | QDLGFFQETK* | 10.8 | (±0.4) | 610.8 > 864.4 | (25) |
| | | (SEQ ID NO: 12) | | | 610.8 > 660.3 | (20) |
| | | | | | 610.8 > 244.1 | (20) |
| I23O1_1(P14902) | 2.5 | VIPTV*FK | 10.3 | (±0.4) | 405.3 > 597.4 | (10) |
| | | (SEQ ID NO: 13) | | | 405.3 > 500.3 | (20) |
| | | | | | 405.3 > 213.2 | (10) |
| I23O1_2(P14902) | 12.5 | YILIPASQQP*K | 10.2 | (±0.4) | 632.4 > 761.4 | (25) |
| | | (SEQ ID NO: 14) | | | 632.4 > 277.2 | (25) |
| | | | | | 632.4 > 503.3 | (10) |
| SYWC(P23381) | 50 | AIDQDPYF*R | 9.6 | (±0.3) | 567.8 > 950.4 | (13) |
| | | (SEQ ID NO: 15) | | | 567.8 > 592.3 | (22) |
| | | | | | 567.8 > 157.1 | (21) |
| UB2L6(P23381) | 25 | ALLLPDQPPYHL*K | 11.4 | (±0.4) | 504.6 > 664.4 | (8) |
| | | (SEQ ID NO: 16) | | | 504.6 > 551.3 | (10) |
| | | | | | 504.6 > 185.1 | (13) |

RT, retention time (min); CE, collision energy (V). Table 2 below shows retention time and transition of the housekeeping control peptide AQUA. (“*” represents ¹³C/¹⁵N stable isotope-label.)

TABLE 2

| | Dose | Amino acid | Retention | | |
|---|---|---|---|---|---|
| Uniprot | (ng) | sequence | time (RT) | Q₁ > Q₃ (CE) | |
| MAPK1_1(P28482) | 12.5 | GQVFDVGP*R | 9.5 (±0.3) | 490.8 > 795.4 | (11) |
| | | (SEQ ID NO: 17) | | 490.8 > 434.3 | (22) |
| | | | | 490.8 > 186.1 | (12) |
| MAPK1_2(P28482) | 25 | LFPNADSK* | 8.4 (±0.3) | 450.2 > 639.3 | (10) |
| | | (SEQ ID NO: 18) | | 450.2 > 261.2 | (8) |
| | | | | 450.2 > 233.2 | (9) |
| PPIB(P23284) | 125 | TVDNFVALATGEK* | 12.3 (±0.4) | 686.9 > 796.5 | (24) |
| | | (SEQ ID NO: 19) | | 686.9 > 697.4 | (22) |
| | | | | 686.9 > 173.1 | (32) |
| ACTB(P60709) | 125 | AGFAGDDAPR* | 7.8 (±0.4) | 493.7 > 711.3 | (16.2) |
| | | (SEQ ID NO: 20) | | 493.7 > 640.3 | (16.2) |
| | | | | 493.7 > 583.3 | (16.2) |

RT, retention time (min);
CE, collision energy (V)

### 2. Preparation of Analysis Sample

The present invention is not limited to the following experimental conditions, but is representative of the following: a macrodissected/cryopulverized tissue powder was added to 100 mM SDS lysis buffer (100 mM Tris-HCl (pH 7.6)/4% SDS/1×protease inhibitor) and crushed using a sonicator (Sonicator 3000), followed by centrifugation at 10° C. and 14,000×g for 10 minutes to collect the supernatant. After protein quantification using a BCA assay kit (Thermo Scientific), peptide digestion was performed using about 150 μg of protein (about 60 μg of peptide) through a FASP (filter aided sample preparation) digestion method. The sample was added to 4% SDT lysis buffer (100 mM Tris-HCl (pH 7.6)/4% SDS/100 mM DTT) so that a total volume was 150 μl, treated at 37° C. for 45 minutes, and then boiled at 90° C. for 10 minutes. The sample cooled to room temperature was dispensed into a 30 K membrane filter (Millipore, MRCFOR030) and then centrifuged at 14,000×g for 40 minutes, after which 200 μl of UA buffer (8 M urea/100 mM Tris-HCl (pH 8.5)) was further added, followed by centrifugation for 40 minutes, and these procedures were repeated three times. Thereafter, 50 mM iodoacetamide in UA buffer was dispensed, allowed to react in the dark for 30 minutes, and centrifuged for another 30 minutes, after which 200 μl of UA buffer was further added, followed by centrifugation for 30 minutes, which was repeated twice. Thereafter, 200 μl of 100 mM tetraammonium bromide (TEAB) was added, followed by centrifugation for 40 minutes, which was repeated twice. About 3 μg of trypsin (amount of trypsin:amount of protein in sample=1:50 (w/w)) was treated with 98% pure custom-made heavy isotopically-labeled internal standard AQUA (hereafter referred to as “heavy peptide”) (Table 1) and allowed to react overnight at 37° C. to obtain a tryptic peptide.

The tryptic peptide was desalted using a C18 Macro Spin Column (Harvard Apparatus, 74-4101). The spin column was activated in a manner in which 500 μl of buffer A (0.1% TFA) was added, centrifugation was performed at 1,000×g for 2 minutes, 500 μl of buffer B (0.1% TFA in 80% acetonitrile) was added, and centrifugation was performed at 1,000×g for 2 minutes. Thereafter, the spin column was equilibrated by performing 3 repetitions of 500 μl each and 2 repetitions of 200 μl each using buffer A under the same conditions. The peptide dissolved in 200 μl of buffer A was added, centrifuged at 400×g for 10 minutes, and eluted, after which the resulting eluate was placed again in the spin column and centrifuged, after which 200 μl of buffer A was added, followed by centrifugation at 800×g for 2 minutes, which was repeated twice, followed by centrifugation at 1,000×g for 2 minutes, which was repeated twice. 200 μl of buffer B was added, followed by centrifugation at 400×g for 2 minutes; 600×g for 2 minutes; and 2,000×g for 2 minutes, and the eluted peptide was dried.

### 3. Nano-Liquid Chromatography (Nano-LC)-Triple Quadrupole

Here, a nano-LC column was used for commercially available chip LC (Agilent Technologies), but any nano-LC column may be utilized in the present invention. In order to perform nano-liquid chromatography (nano-LC)-triple quadrupole using the AQUA peptide of Tables 1 and 2, fractionation (RPLC fractionation) of the sample was performed as follows.

For organic solvents of the mobile phase, 10 mM TEAB as solvent A and 10 mM TEAB in acetonitrile as solvent B were used, and RPLC fractionation was performed for 115 minutes at a pH of 7.5 by connecting a Xbridge C18 analytical column (4.6 mm×250 mm, 130 A, 5 μm) to HPLC (1260 Infinity HPLC system (Agilent Technologies)) [flow rate, 500 μl/min; 0% solvent B (10 min), 0→5% solvent B (10 min), 5→35% solvent B (60 min), 35→70% solvent B (15 min), 70% solvent B (10 min), 70→0% solvent B (10 min)]. 96 fractions were obtained at 1-minute intervals for 15 to 110 minutes, followed by non-contiguously concatenated fractionation to afford 12 fractions, which were then dried.

Each fractionated peptide was dissolved in 20 μl of 0.1% formic acid, and then 4 μl thereof was injected into a 1200 nanoflow pump (Agilent Technologies) using an autosampler. The injected peptide was separated through a large-capacity microfluidic chip (G4240-62010) connected to a 6490 triple quadrupole mass spectrometer (Agilent Technologies) with a dedicated chip LC interface, in which the chip LC was composed of a Zorbax-SB C-18, 300 A 5 μm-particle 150 mm×75 μm analytical column and a 160 nl trapping column.

Using 0.1% formic acid in water as solvent A and 0.1% formic acid in methanol as solvent B for the mobile phase, nano-liquid chromatography was performed for 30 minutes per fraction. Analytical chromatographic conditions were as follows: a flow rate of 3 μl/min for a capillary pump [5→95% solvent B (15 min), 95→95% solvent B (8 min), 95→5% solvent B (2 min)], and a flow rate of 0.3 μl/min for a nanoflow pump [5→95% solvent B (14 min), 95→95% B (7 min), 95→5% solvent B (0.1 min)].

The chip LC operated in a forward flush mode, and the triple quadrupole mass spectrometer was set under conditions of a dry gas temperature of 250° C., a dry gas flow rate of 15 L/min, a capillary voltage of 2100 V (positive), ΔEMV of 0 V, a fragmentor voltage of 380 V, a cell accelerator voltage of 5 V, and a cycle time of 0.79 cycles/sec. Under these conditions, transition of AQUA and target peptide (endogenous peptide) was analyzed, but specific experimental conditions of the present invention are not limited to the foregoing.

Table 3 below shows results of analyzing the target peptide (light endogenous peptide) through MRM quantification.

TABLE 3

| Peptide | Uniprot | Amino acid sequence | RT | [symbol] |
|---|---|---|---|---|
| [symbol] | [symbol] | [symbol] | 10 (±0.3) | [symbol] |
| | | (SEQ ID NO: 1) | | [symbol] |
| | | | | [symbol] |
| [symbol] | [symbol] | [symbol] | [symbol] | [symbol] |
| | | (SEQ ID NO: 2) | | [symbol] |
| | | | | [symbol] |
| [symbol] | [symbol] | [symbol] | 10.4 (±0.4) | [symbol] |
| | | (SEQ ID NO: 3) | | [symbol] |
| | | | | [symbol] |
| [symbol] | [symbol] | [symbol] | 8.5 (±0.2) | [symbol] |
| | | (SEQ ID NO: 4) | | [symbol] |
| | | | | [symbol] |
| [symbol] | [symbol] | [symbol] | 8.3 (±0.5) | [symbol] |
| | | (SEQ ID NO: 5) | | [symbol] |
| | | | | [symbol] |
| [symbol] | [symbol] | [symbol] | 7.8 (±0.4) | [symbol] |
| | | (SEQ ID NO: 6) | | [symbol] |
| | | | | [symbol] |
| [symbol] | [symbol] | [symbol] | 9.6 (±0.4) | [symbol] |
| | | (SEQ ID NO: 7) | | [symbol] |
| | | | | [symbol] |
| [symbol] | [symbol] | [symbol] | [symbol] | [symbol] |
| | | (SEQ ID NO: 8) | | [symbol] |
| | | | | [symbol] |
| [symbol] | [symbol] | [symbol] | 8 (±0.4) | [symbol] |
| | | (SEQ ID NO: 9) | | [symbol] |
| | | | | [symbol] |
| [symbol] | [symbol] | [symbol] | 7 (±0.2) | [symbol] |
| | | (SEQ ID NO: 10) | | [symbol] |
| | | | | [symbol] |
| [symbol] | [symbol] | [symbol] | 9.4 (±0.3) | [symbol] |
| | | (SEQ ID NO: 11) | | [symbol] |
| | | | | [symbol] |
| [symbol] | [symbol] | [symbol] | 10.8 (±0.4) | [symbol] |
| | | (SEQ ID NO: 12) | | [symbol] |
| | | | | [symbol] |
| [symbol] | [symbol] | [symbol] | 10.3 (±0.4) | [symbol] |
| | | (SEQ ID NO: 13) | | [symbol] |
| | | | | [symbol] |
| [symbol] | [symbol] | [symbol] | 10.2 (±0.4) | [symbol] |
| | | (SEQ ID NO: 14) | | [symbol] |
| | | | | [symbol] |
| [symbol] | [symbol] | [symbol] | 9.6 (±0.3) | [symbol] |
| | | (SEQ ID NO: 15) | | [symbol] |
| | | | | [symbol] |
| [symbol] | [symbol] | [symbol] | 11.4 (±0.4) | [symbol] |
| | | (SEQ ID NO: 16) | | [symbol] |
| | | | | [symbol] |

[symbol]

indicates data missing or illegible when filed

Table 4 below shows a housekeeping control peptide (light endogenous peptide) that is quantified together for normalization of the target peptide upon MRM analysis.

TABLE 4

| Peptide | Uniprot | Amino acid sequence | Retention time (RT) | Q₁ > Q₃ |
|---|---|---|---|---|
| MAPK1_1 | P28482 | GQVFDVGPR | 9.5 (±0.3) | 487.8 > 789.4 |
| | | (SEQ ID NO: 17) | | 487.8 > 428.3 |
| | | | | 487.8 > 186.1 |
| MAPK1_2 | P28482 | LFPNADSK | 8.4 (±0.3) | 446.2 > 631.3 |
| | | (SEQ ID NO: 18) | | 446.2 > 261.2 |
| | | | | 446.2 > 233.2 |
| PPIB | P23284 | TVDNFVALATGEK | 12.3 (±0.4) | 682.9 > 788.5 |
| | | (SEQ ID NO: 19) | | 682.9 > 689.4 |
| | | | | 682.9 > 173.1 |
| ACTB | P60709 | AGFAGDDAPR | 7.8 (±0.4) | 488.7 > 701.3 |
| | | (SEQ ID NO: 20) | | 488.7 > 630.3 |
| | | | | 488.7 > 573.3 |

RT, retention time (min); MRM raw data was transferred to the Skyline program (software), and the chromatographic peak AUC of Q₃ transition was absolutely quantified using a standard curve (in this example, quantification was performed on the basis of the transition with the largest AUC).

For peptides identified in multiple fractions, individual fractions were summed. Standard curves were created in a manner in which MRM was performed on, using an E. coli protein (tryptic digest of E. coli lysate) as a matrix, a fixed amount of synthetic peptide (heavy AQUA peptide; Tables 1 and 2) and various proportions of synthetic target peptide (light peptide; endogenous peptide; Tables 3 and 4), resulting in regression of the light/heavy ratio for the target peptide (pg) (FIGS. 1a to 1g).

### 4. Prediction of Effect of Targeting Agent Through MRM Analysis in Tumor Tissue/Normal Tissue

The present invention is not limited to the following analysis method, but typically analyzes MRM data as follows. For cases in which MRM may be performed on both tumor tissue isolated from a pre-treatment patient before immunotherapy and adjacent normal tissue at the same time point/organ (tissue type), clinical response was predicted by performing analysis in which the relative expression level (ratio) of tumor tissue normalized with a housekeeping control peptide compared to normal tissue was treated as a representative value (FIG. 2 and FIGS. 5a to 5h).

As in Equation 1, the amount of each peptide (endogenous peptide; light peptide) based on the spike-in AQUA peptide (heavy peptide) was calculated using a standard curve (FIGS. 1a to 1g).

[Equation 1]

[symbol]

Then, as in Equation 2, the amount of the target peptide (endogenous peptide; light peptide) in Table 3 was normalized by being divided by the amount of the housekeeping control peptide, and the relative expression level was represented as the ratio between the relevant tumor and normal (ratio_{tumor/normal}) (for peptides for which MRM identification was not possible, calculation was performed by substituting the lower limit of quantification (LLOQ)).

For ERBB2, EGFR, FGFR2, MET, and PD-L1, a representative value was taken with the indicator (peptide having high average AUC) set by the analyst among two peptides.

$$Ratio \text{?} = \frac{amount \text{?}}{amount \text{?}} \times \text{?} [ \frac{amount \text{?}}{amount \text{?}} ] , \qquad [ Equation 2 ]$$

$$\text{?} \text{indicates text missing or illegible when filed}$$

Based on results of ratio_{tumor/normal}, it is possible to predict whether the target protein is overexpressed and clinical efficacy of the targeting agent under the following criteria.

As such, the higher the expression level of the target peptide (target protein), the higher the drug response, and thus the cutoff of the ratio, which is the criterion for selecting each targeting agent, is not limited to the following numerical value.

Based on results of analyzing the ratio_{tumor/normal} values in examples of the present invention, the efficacy of the targeting agent was predicted to be high as shown in Table 5 below.

TABLE 5

| Ratio_{tumor/normal} conditions | Prediction results |
|---|---|
| When EGFR ratio_{tumor/normal} > 5 | Efficacy of EGFR- |
| | targeting agent being |
| | predicted to be high |
| When FGFR2 ratio_{tumor/normal} > 5 | Efficacy of FGFR- |
| | targeting agent being |
| | predicted to be high |
| When MET ratio_{tumor/normal} > 5 | Efficacy of MET- |
| | targeting agent being |
| | predicted to be high |
| When ERBB2 ratio_{tumor/normal} > 5 | Efficacy of HER2- |
| | targeting agent being |
| | predicted to be high |
| When PD-L1 ratio_{tumor/normal} > 3 or quantification | Efficacy of |
| is possible only in tumor tissue, or when average | immunotherapy being |
| of tumor/normal (T/N) ratio_{I2301}_{_{—}}₁, T/N | predicted to be high |
| ratio_{I2301}_{_{—}}₂, T/N ratio_{TAP2}_{_{—}}₁, T/N ratio_{TAP2}_{_{—}}₂, | |
| T/N ratio_{SYWC}, and T/N ratio_{UB2L6} > 5 | |

### 5. Prediction of Effect of Targeting Agent Through MRM Analysis in Tumor Tissue

Depending on the clinical situation, there may be cases where only pre-treatment tumor tissue is available for MRM analysis and adjacent normal tissue at the same time point/organ (tissue type) is not available. As such, as shown in FIG. 3, clinical response was capable of being predicted based on housekeeping control-normalized MRM quantitative values of five proteins such as EGFR, FGFR2, ERBB2, MET, and PD-L1 in the pre-treatment tumor sample.

As in Equation 3, the amount of the target peptide (endogenous peptide; light peptide) was normalized by being divided by the amount of housekeeping control peptide.

[Equation ]

[symbol]

Also, how high the normalized tissue amount of each tumor sample was compared to the average normalized tissue amount of all-tumor MRM database was determined. The all-tumor MRM database was composed of values calculated by applying the present invention to the same protocol for each tissue type. The median z value obtained by normalization with the housekeeping control peptide was used for final determination.

Typically, when the normalized tissue amount shows a high value exceeding the standard deviation of +1.96 from the average normalized amount of all-tumor MRM database (5% outlier), the relevant peptide is evaluated to be overexpressed, based on which the efficacy of the relevant agent may be predicted to be high.

Briefly, when there is a peptide with a value of Tumor normalized tissue amount>Average normalized tissue amount+(1.96×SD_{normalized tissue amount}), it corresponds to z_{normalized tissue amount}>1.96, so clinical efficacy of targeted therapy targeting the relevant protein is predicted to be high (Table 6).

As such, the higher the expression level of the target protein, the higher the drug response, and thus the cutoff of the z value, which is the criterion for selecting each targeting agent, is not limited to Table 6 below.

TABLE 6

| Target peptide z_{normalized tissue amount} | |
|---|---|
| conditions in tumor tissue | Prediction results |
| When EGFR z_{normalized tissue amount} in | Efficacy of EGFR-targeting agent |
| tumor tissue > 1.96 | being predicted to be high |
| When FGFR2 z_{normalized tissue amount} in | Efficacy of FGFR-targeting agent |
| tumor tissue > +1.96 | being predicted to be high |
| When MET z_{normalized tissue amount} in | Efficacy of MET-targeting agent |
| tumor tissue > +1.96 | being predicted to be high |
| When ERBB2 z_{normalized tissue amount} in | Efficacy of HER2-targeting agent |
| tumor tissue > +1.96 | being predicted to be high |
| When PD-L1 z_{normalized tissue amount} in | Efficacy of PD-1/PD-1 antibody |
| tumor tissue > +1.96 | being predicted to be high |

Meanwhile, when analyzing the z value of the normalized tissue amount of the tumor sample compared to the normalized tissue amount of all-tumor MRM database, the normalized tissue amount corrected for tumor purity in samples for which tumor purity information was obtained by genome analysis (NGS) on the same tissue sample was used as all-tumor MRM database. The z value of the purity-adjusted amount was determined by selecting the median z value normalized with the housekeeping control peptide as a representative value, and in cases of exceeding the standard deviation of +1.96 from the average purity-adjusted amount of all-tumor MRM database, the efficacy of the targeting agent was predicted to be high.

6. Correction of Tumor Purity

In the method of predicting the effect of the targeting agent through MRM analysis in tumor tissue, in order to increase precision when evaluating how high the normalized tissue amount of each tumor sample is compared to the average normalized tissue amount of all-tumor MRM database, the all-tumor MRM database may be composed of data corrected for tumor purity as in Equation 4 below. The same tumor tissue sample on which MRM is performed is corrected when the tumor purity value may be determined by a method such as NGS, etc., so that clinical drug response may be predicted based on the purity-adjusted MRM value compared to the all-tumor MRM database.

[Equation 4]

[symbol]

When the median z_{purity-adjusted amount} value obtained by normalization with the housekeeping control peptide in this way exceeds the standard deviation of +1.96 from the average purity-adjusted amount of all-tumor MRM database (z_{purity-adjusted amount}>1.96; 5% outlier), the efficacy of the targeting agent may be predicted to be high (in the present invention, the cutoff of the z value, which is the criterion for selecting the targeting agent, is not limited to this numerical value).

### [Example 1]Clinical Response to Immunotherapy and Comparative Verification

Pre-treatment frozen gastric cancer and adjacent normal tissue samples of a male gastric cancer patient with microsatellite instability-high (MSI-H) who showed partial response to anti-PD-1 alone as immunotherapy (patient 1) and female (patient 2) and male (patient 3) gastric cancer patients who did not respond to the same anti-PD-1 alone were analyzed by MRM and clinical response to the immunotherapy was predicted.

When the ratio_{tumor/normal} value of each of the PD-L1 (CD274), TAP2 (TAP2), I23O1 (IDO1), SYWC (WARS1), and UB2L6 (UBE2L6) peptides obtained using Equation 1 satisfied at least one of the following two conditions, efficacy of the immunotherapy was predicted to be high.

### Condition 1:

PD-L1 (CD274) shows ratio_{tumor/normal}>3, or is quantifiable only in tumor tissue and not quantifiable in adjacent normal tissue.

### Condition 2:

An average ratio_{tumor/normal}value of six peptides representing TAP2 (TAP2), I23O1 (IDO1), SYWC (WARS1), and UB2L6 (UBE2L6) proteins (average of T/N ratio_{I23O1_1}, T/N ratio_{I23O1_1}, T/N ratio_{TAP2_1}, T/N ratio_{TAP2_2}, T/N ratio_{SYWC}, and T/N ratio_{UB2L6}) exceeds 5.

The housekeeping control-normalized PD-L1 ratio_{tumor/normal} quantitative values of patients 1 to 3 were all 0, and the housekeeping control-normalized immunoprotein ratio_{tumor/normal} quantitative values of patients 1 to 3 are shown in Table 7 below.

TABLE 7

| Normalized | Patient 1: PD-1 | Patient 2: PD-1 | Patient 3: PD-1 |
|---|---|---|---|
| peptide | therapy clinical | therapy clinical | therapy clinical |
| quantitative | response | non-response | non-response |
| value | tumor/normal | tumor/normal | tumor/normal |
| I23O1_1(IDO1) | 41.9 | 6.4 | 1.0 |
| I23O1_2(IDO1) | 1.0 | 1.0 | 1.0 |
| TAP2_1 | 5.6 | 1.3 | 3.4 |
| TAP2_2 | 1.0 | 1.0 | 1.0 |
| UB2L6 (UBE2L6) | 3.1 | 3.0 | 7.1 |

As described above, patient 1 had microsatellite instability-high (MSI-H) gastric cancer. MSI-H gastric cancer is well known for immunoprotein overexpression and high response to immunotherapy, and patient 1 actually showed a good clinical result of partial response to anti-PD-1 alone as the immunotherapy. For patient 1 who had clinical response to the immunotherapy, the average ratio_{tumor/normal} value of six peptides for immunoproteins (I23O1, TAP2, UB2L6, and SYWC) was 9.7 (Table 7), and although the PD-L1 value was 0, one of the two conditions required to be predicted to show good response to the immunotherapy according to the present invention was satisfied, confirming that the prediction method according to the present invention showed results consistent with actual clinical response to the immunotherapy.

For patient 2 and patient 3 who had no clinical response to the immunotherapy, the average immunoprotein values were 2.2 and 2.6, respectively (Table 7), and the PD-L1 value was 0, indicating that neither of the two conditions predicted to respond to the immunotherapy according to the present invention was satisfied.

Therefore, the prediction method according to the present invention was confirmed to show results consistent with actual clinical response to the immunotherapy.

### [Example 2]Comparison Depending on Immunotherapeutic Response Predictive Indicator (EBV, MSI Positive/Negative)

The MRM prediction results of the present invention were compared with EBV and MSI status, which can be said to be representative predictive indicators of clinical response to immunotherapy (PD-1 antibody).

Although it was not possible to confirm clinical response by direct administration of immunotherapy as with patients 1 to 3, in gastric cancer patients including an MSI-H (positive) patient with activated immunity (patient 4), an EBV/MSI-negative patient (patient 5), and an EBV-positive patient with activated immunity (patient 6), overexpression of immunoproteins was compared and evaluated according to the prediction method of the present invention (Table 8).

Table 8 below shows the housekeeping control-normalized PD-L1 ratio_{tumor/normal} quantitative values in patients 4 to 6.

TABLE 8

| | | Patient 5: | |
|---|---|---|---|
| | Patient 4: | MSI/EBV- | Patient 6: |
| Normalized peptide | MSI-H | negative | EBV-positive |
| quantitative value | tumor/normal | tumor/normal | tumor/normal |
| PD-L1_1(CD274) | 0 | 0 | 180.6 |

Table 9 below shows the housekeeping control-normalized immunoprotein ratio_{tumor/normal} quantitative values in patients 4 to 6.

TABLE 9

| | | Patient 5: | |
|---|---|---|---|
| | Patient 4: | MSI/EBV- | Patient 6: |
| Normalized peptide | MSI-H | negative | EBV-positive |
| quantitative value | tumor/normal | tumor/normal | tumor/normal |
| I23O1_1(IDO1) | 37.6 | 1.0 | 32.6 |
| I23O1_2(IDO1) | 16.3 | 1.0 | 14.9 |
| TAP2_1 | 1.0 | 1.0 | 4.3 |
| TAP2_2 | 1.0 | 1.0 | 1.0 |
| UB2L6(UBE2L6) | 1.0 | 1.0 | 10.4 |
| SYWC(WARS1) | 15.1 | 2.1 | 11.6 |
| Average | 12.0 | 1.2 | 12.5 |
| ratio_{tumor/normal} | | | |

For patient 4 (MSI-H) and patient 6 (EBV positive) who are expected to clinically respond to the immunotherapy, the average ratio_{tumor/normal} values of six peptides for immunoproteins (I23O1, TAP2, UB2L6, and SYWC) were determined to be 12.0 and 12.5, respectively (Table 9). Although the PD-L1 ratio_{tumor/normal} value exceeded the reference value of 3 only in patient 6 (Table 8), patients 4 and 6 satisfied at least one of the two conditions required to be predicted to show good response to the immunotherapy.

For patient 5 who is expected not to clinically respond to immunotherapy (both MSI/EBV negative), the average value of immunoproteins was also 1.2 (Table 9), and the PD-L1 ratio_{tumor/normal} value was also 0 (Table 8), so it was predicted that there was no overexpression of immunoproteins. As described above, the results of the present invention for patients 4 to 6 were consistent with the immunotherapeutic response predictive indicators (MSI/EBV).

### [Example 3] Determination of Immunoprotein Overexpression Through MRM Analysis of Only Tumor Tissue

Like patient 4 and patient 6, EBV- and MSI-positive patients with activated immunity were compared with gastric cancer tissue MRM data of patient 5 who is negative for both EBV and MSI. Here, MRM analysis was performed only with tumor tissue results, excluding normal tissue results.

MRM analysis was performed in the same manner as in Example 1, and how high the normalized tissue amount (Equation 3) of immunoprotein was compared to the average normalized tissue amount of all-tumor MRM database was evaluated in tumor samples.

When MRM data obtained from 11 gastric cancer samples was used as all-tumor MRM database, the normalized amount of the relevant tumor compared to the normalized amount of all-tumor MRM database was calculated as z_{normalized tissue amount} of each peptide. As such, the z value of the normalized amount was the median z value obtained by normalization with a housekeeping control peptide (Table 10).

Table 10 below shows results of median z_{normalized tissue amount} of PD-L1 in gastric cancer tissues of patients 4 to 6.

TABLE 10

| | | Patient 5: | |
|---|---|---|---|
| | | MSI/EBV- | Patient 6: |
| | Patient 4: | negative | EBV-positive |
| | MSI-H PD-L1 | PD-L1 | PD-L1 |
| | z_{normalized tissue amount} | z_{normalized tissue amount} | z_{normalized tissue amount} |
| Median | z = 0.28 | z = −0.31 | z = 2.19 |
| z value | | | |

As described above, in this example, the efficacy of the PD-1/PD-1 antibody was predicted to be high when PD-L1 z_{normalized tissue amount} in tumor tissue>+1.96. For EBV-positive patient 6, the median z_{normalized tissue amount} value of PD-L1 was 2.19, which was predicted to have high clinical response to anti-PD-1/PD-1 antibody, and for EBV/MSI-negative patient 5, the median z_{normalized tissue amount} value of PD-L1 was −0.31, which was predicted to have low response. Ultimately, it was confirmed that the inventive results were generally consistent with clinical findings.

### [Example 4] Tumor Purity Correction in MRM Analysis of Only Tumor Tissue

With a concept similar to CPS (combined positive score) serving as a stratification factor in gastric cancer clinical trials of pembrolizumab/nivolumab, the above analysis data was corrected with tumor purity of tumor tissue so that PD-L1 protein quantification became more precise.

Tumor purity was calculated by analyzing the data resulting from WGS of genomic DNA isolated from the same tissue as the tissue on which MRM was performed. As analyzed by the z value of the normalized tissue amount of the tumor sample compared to the average normalized tissue amount of all-tumor MRM database, the purity-adjusted tissue amount in 10 samples (median purity, 0.42) for which tumor purity information was obtained was used as all-tumor MRM database (Table 11).

TABLE 11

| | | Patient 5: | |
|---|---|---|---|
| | Patient 4: | MSI/EBV- | Patient 6: |
| | MSI-H | negative | EBV-positive |
| Tumor purity (WGS) | 0.46 | 0.44 | 0.29 |
| Median z_{purity-adjusted amount} | z = −0.36 | z = −0.33 | z = 2.58 |
| of PD-L1 | | | |

When the z_{purity-adjusted amount} corrected by Equation 4 exceeded 1.96, the relevant target peptide was determined to be overexpressed. For EBV-positive patient 6, the median z_{purity-adjusted amount} value of PD-L1 was 2.58, which was determined to be PD-L1 overexpression. For EBV/MSI-negative patient 5, the median z_{purity-adjusted amount} value of PD-L1 was −0.33, which was determined to mean that there was no PD-L1 overexpression, indicating that the present invention was consistent with clinical information. Compared with patient 6 in Example 3 (z=2.19), the results after tumor purity correction (z=2.58) can be regarded as more precise.

### [Example 5] Prediction of Efficacy of FGFR Inhibitor and MET Inhibitor

MRM was performed in the same manner as in the previous examples for two gastric cancer cell lines having a significant difference in response depending on the type of immunotherapy.

According to results of 72-hour MTT experiment by the inventors, IC₅₀ of rogaratinib as an FGFR inhibitor was 30.977 nM in a KATO-III cell line, which is an FGFR2-amplified cell line, but exceeded 40 nM (>40 nM) in SNU-5 cells, confirming low response to the FGFR inhibitor. Response to the FGFR inhibitor was significantly higher in KATO-III, which is an FGFR2-amplified cell line, than in SNU-5. In existing literature report, it is known that response to dovitinib as an FGFR inhibitor is relatively higher in an FGFR2-amplified cell line KATO-III than in a SNU-5 cell line.

In contrast, according to results of 72-hour MTT experiment by the inventors, IC50 of crizotinib as a MET inhibitor was 22.61 nM in a KATO-III cell line, but was 17.53 nM in SNU-5, which is a MET-amplified cell line, indicating that response to the MET inhibitor was significantly higher in SNU-5 than in KATO-III. For response of gastric cancer cell lines to the MET inhibitor, it has also been reported in existing literature that response to the MET inhibitor is relatively higher in SNU-5 than in KATO-III, which was consistent with the experimental results.

Table 12 below shows the median z_{normalized tissue amount} value of housekeeping control-normalized FGFR2.

TABLE 12

| | KATO-III | SNU-5 |
|---|---|---|
| | z_{normalized tissue amount} | z_{normalized tissue amount} |
| FGFR2_1 | z = 2.25 | z = −0.43 |
| FGFR2_2 | z = 2.85 | z = −0.32 |

In the present invention, the median z_{normalized tissue amount} value of housekeeping control-normalized FGFR2 was z=−0.43 or z=−0.32 in SNU-5, and z=2.25 or z=2.85 in KATO-III, indicating that response to the FGFR inhibitor was predicted to be high in KATO-III with z_{normalized tissue amount}>1.96, which was consistent with the experimental data of the inventors in which IC₅₀ of rogaratinib as the FGFR inhibitor was relatively low in the KATO-III cell line and thus response to the FGFR inhibitor was relatively high.

For reference, in global proteomic profiling data by the inventors, the FGFR2 reporter ion intensity ratio was 3.27 for KATO-III and 1.03 for SNU-5 compared to cyclophilin B. Therefore, the results of this example exhibited a high correlation with global proteomic profiling data.

Table 13 below shows the median z_{normalized tissue amount} value of housekeeping control-normalized MET.

TABLE 13

| | KATO-III | SNU-5 |
|---|---|---|
| | z_{normalized tissue amount} | z_{normalized tissue amount} |
| MET_2 | z = −0.19 | z = 2.77 |
| MET_4 | z = 0.58 | z = 2.26 |

As is apparent from Table 13, the median z_{normalized tissue amount} value of housekeeping control-normalized MET was z=0.19 or z=0.58 in KATO-III, and z=2.77 or z=2.26 in SNU-5, indicating that response to the MET inhibitor was predicted to be high in SNU-5 with z_{normalized tissue amount}>+1.96, which was consistent with the experimental data of the inventors in which IC₅₀ of crizotinib as the MET inhibitor was relatively low in SNU-5 and thus response to the MET inhibitor was relatively high.

For reference, in global proteomic profiling data by the inventors, the MET reporter ion intensity ratio was measured to be 0.97 for KATO-III and 3.44 for SNU-5, which was consistent with the results of the present invention.

### [Example 6] Evaluation of Expression Level of HER2 (ERBB2) Peptide

High clinical response to a HER2 (ERBB2)-targeting agent in the presence of ERBB2 gene amplification has been clinically confirmed by existing clinical trials. According to research results by the inventors, the protein expression level of ERBB2 and signaling system and clinical drug response are directly proportional. Therefore, the level of ERBB2 peptide overexpression is analyzed and may be used to predict clinical response to the HER2-targeting agent.

MRM of the present invention was applied to gastric cancer patient 4 with ERBB2 gene amplification on whole genome sequencing and ERBB2 protein overexpression (tumor/normal reporter ion intensity ratio, 18.36) on global proteomic profiling data, and the results thereof were compared with results of the other gastric cancer without ERBB2 gene amplification.

Gastric cancer in patient 4 was a sample with focal gene amplification of ERBB2 locus, log2 ratio 3.5, copy number 23 as confirmed by whole genome sequencing, and was compared and analyzed with patients 1, 3, and 5 without ERBB2 gene amplification. A comparison was made with the housekeeping control-normalized tumor/normal HER2 protein ratio according to Equations 1 and 2.

Table 14 below shows the median ratio_{tumor/normal} value of housekeeping control-normalized HER2.

TABLE 14

| | | Patient 1 | Patient 2 | Patient 3 | Patient 4 | Patient 6 |
|---|---|---|---|---|---|---|
| | Patient 5 | tumor/normal | tumor/normal | tumor/normal | tumor/normal | tumor/normal |
| | tumor/normal | No | No | No | No | No |
| | Overexpression/ | overexpression/ | overexpression/ | overexpression/ | overexpression/ | overexpression/ |
| | clinical | no | no | no | no | no |
| | response | response | response | response | response | response |
| | prediction | prediction | prediction | prediction | prediction | prediction |
| ERBB2_ | 72.0 | 1.7 | 1.8 | 0.7 | 1 | 1 |
| 4 | | | | | | |
| ERBB2_ | 700.2 | 1 | 1 | 1.6 | 1 | 1 |
| 5 | | | | | | |

As is apparent from Table 14, the median value of the housekeeping control-normalized ERBB2 peptide tumor/normal ratio in patient 5 diagnosed with gastric cancer with ERBB2 gene amplification and protein overexpression on global proteomic profile was 72.0 or 700.2.

Only in this patient was the efficacy of the HER2-targeting agent predicted to be superior due to HER2 protein overexpression (ratio_{tumor/normal}>5). In the other gastric cancer patients without ERBB2 gene amplification, the housekeeping control-normalized ERBB2 peptide tumor/normal ratio (ratio_{tumor/normal})<5 was confirmed.

### [Example 7] Evaluation of HER2 Overexpression through MRM Analysis of Only Tumor Tissue

Since clinical response to the HER2-targeting agent is high in the presence of ERBB2 gene amplification as described above, the present invention was applied to patient 4 of Example 6 only with tumor tissue MRM data without normal tissue MRM data.

*The normalized amount of the relevant tumor compared to the normalized amount of all-sample MRM database was calculated as the z value of each peptide, using MRM data of 11 gastric cancer patients as all-sample MRM database, and the z value of the normalized amount was taken as the median z value of the housekeeping control-normalized amount. In this example, as in the previous examples, when the normalized tissue amount for HER2 (ERBB2) showed a high value exceeding the standard deviation of +1.96 from the average normalized amount of all-sample MRM database (5% outlier), HER2 (ERBB2) peptide was evaluated to be significantly overexpressed. Table 15 below shows results of z_{normalized tissue amount} of housekeeping control-normalized ERBB2.

TABLE 15

| | | Patient 1 | Patient 2 | Patient 3 | Patient 4 | Patient 6 |
|---|---|---|---|---|---|---|
| | Patient 5 | tumor/normal | tumor/normal | tumor/normal | tumor/normal | tumor/normal |
| | tumor/normal | No | No | No | No | No |
| | Overexpression/ | overexpression/ | overexpression/ | overexpression/ | overexpression/ | overexpression/ |
| | clinical | no | no | no | no | no |
| | response | response | response | response | response | response |
| | prediction | prediction | prediction | prediction | prediction | prediction |
| ERBB2_ | 2.85 | −0.31 | −0.31 | −0.32 | −0.07 | −0.44 |
| 4 | | | | | | |
| ERBB2_ | 2.85 | −0.33 | −0.33 | −0.28 | −0.55 | −0.37 |
| 5 | | | | | | |

As is apparent from Table 15, the median value of the normalized tissue ERBB2 amount in gastric cancer of patient 5 with ERBB2 gene amplification and HER2 protein overexpression (overexpression on global proteomic profile) was z=2.85 or z=2.85, and only this sample was predicted to have high clinical efficacy of the HER2-targeting agent due to HER2 protein overexpression (z_{normalized tissue amount}>1.96). In contrast, the other gastric cancer patients (patients 1, 2, 3, 4, and 6) without ERBB2 gene amplification showed z_{normalized tissue amount}<1.96, which was predicted to have no response.

### [Example 8] Prediction of Clinical Response to HER2 (ERBB2)-Targeting Agent

The present invention was applied to gastric cancer tissues isolated from pre-treatment of two male gastric cancer patients (patients 7 and 8) who were both positive on HER2 immunostaining (3+, 2+) and received combination therapy with trastuzumab (trade name: Herceptin), which is a HER2-targeting agent.

Both patients received combination therapy with trastuzumab, which is a HER2-targeting agent, as first-line anticancer therapy for metastatic gastric cancer, but patient 7 showed partial response, and patient 8 showed progressive disease at the time of first response evaluation and treatment was discontinued, indicating good/poor clinical response results in respective cases.

In the same manner as in Example 7, clinical validity of the present invention for the ERBB2 (HER2)-targeting agent was confirmed only with MRM data of pre-treatment frozen tumor tissue without MRM data of normal tissue. The normalized ERBB2 tissue amount of the tumor sample compared to the average normalized ERBB2 tissue amount of all-tumor MRM database for 11 gastric cancer samples, namely the normalized amount of the relevant tumor compared to the normalized amount of all-tumor MRM database was calculated as the z value (the z value of the normalized amount was the median z value of the housekeeping control-normalized amount). In this example, the efficacy of the ERBB2 (HER2) inhibitor was predicted to be high when the normalized tissue amount for ERBB2 showed a high value exceeding the standard deviation of +1.96 from the average normalized amount of all-tumor MRM database.

Table 16 below shows results of z_{normalized tissue amount} of housekeeping control-normalized ERBB2.

TABLE 16

| | Patient 7 | Patient 8 |
|---|---|---|
| | tumor | tumor |
| | Clinical drug response | Clinical non-response |
| ERBB2_4 | z = 76.0 | z = 0.43 |
| ERBB2_5 | z = 67.9 | z = 0.84 |

As is apparent from Table 16, the median value of the normalized tissue ERBB2 amount in patient 7 who had clinical response to ERBB2 antibody therapy was z=76.0 or z=67.9, which was predicted to have high clinical efficacy of the ERBB2-targeting agent (z_{normalized tissue amount}>1.96) whereas the median value of the normalized tissue ERBB2 amount in gastric cancer of patient 8 who had no clinical response to the same therapy was z=0.43 or z=0.84, which was predicted to have low clinical efficacy of the ERBB2-targeting agent (z_{normalized tissue amount}<1.96). Both of the above two cases were determined to be HER2-positive gastric cancer based on the pathological examination results and received trastuzumab administration, but only patient 7 was judged to be expected to be efficacious based on the results of the present invention.

Therefore, the present invention exhibited superior results consistent with actual clinical response compared to typical pathological tests, ultimately demonstrating the clinical validity of the present invention for predicting clinical response to the targeting agent.

### [Example 9] Verification of Prediction of Clinical Response to EGFR-Targeting Agent

*Clinical response to an EGFR-targeting agent in a male gastric cancer patient with EGFR protein overexpression on immunostaining due to strong EGFR 3+(range, 0˜3+) positive on EGFR immunostaining and a female gastric cancer patient with EGFR negative on immunostaining was compared with MRM results of the present invention.

Patient 9 was subjected to combination therapy with EGFR antibody, indicating a good clinical result of partial response. Patient 2 was subjected to combination therapy with the same EGFR antibody, but showed poor clinical response of disease progression at the time of first response evaluation.

Table 17 below shows housekeeping control-normalized EGFR ratio_{tumor/normal} quantitative values.

TABLE 17

| | Patient 9: EGFR antibody | Patient 2: EGFR antibody |
|---|---|---|
| | therapy clinical response | therapy clinical non- |
| | tumor/normal | response tumor/normal |
| EGFR_2 | 159.1 | 1 |
| Ratio_{tumor/normal} | | |
| EGFR_4 | 13.6 | 1 |
| Ratio_{tumor/normal} | | |

As is apparent from Table 17, the median value of the housekeeping control-normalized tumor/normal EGFR ratio in patient 9 who had clinical response to EGFR antibody therapy was 159.1 or 13.6, indicating the results of prediction in which targeted therapy was recommended due to EGFR protein overexpression (EGFR ratio_{tumor/normal}>5). On the other hand, the median value of the housekeeping control-normalized EGFR tumor/normal ratio in patient 2 who had no clinical response to the same EGFR antibody therapy was 1 or 1, indicating ratio_{tumor/normal}<5. These results show the clinical validity of the present invention for predicting clinical response to the EGFR-targeting agent.

### [Example 10] Verification of Prediction of Clinical Response to EGFR-Targeting Agent Only with Tumor Tissue

For the same patients in Example 9, the present invention was verified only with MRM data of pre-treatment tumor tissue without MRM data of normal tissue. As in the previous examples, the housekeeping control-normalized amount of the relevant tumor compared to the housekeeping control-normalized amount of all-tumor MRM database for 11 gastric cancer samples was calculated as a z value, and the z value of the normalized amount was determined by selecting the median z value of the housekeeping control-normalized amount as a representative value. In this example, when the normalized tissue amount for EGFR showed a high value exceeding the standard deviation of +1.96 from the average normalized amount of all-tumor MRM database (5% outlier), the EGFR peptide was evaluated to be significantly overexpressed, based on which the efficacy of the EGFR inhibitor was predicted to be high.

Table 18 below shows the normalized tissue amount of housekeeping control-normalized EGFR and the median z_{normalized tissue amount}.

TABLE 18

| | Patient 9: EGFR antibody | Patient 2: EGFR antibody |
|---|---|---|
| | therapy clinical response | therapy clinical non- |
| | tumor | response tumor |
| EGFR_2 | z = 2.38 | z = −0.57 |
| EGFR_4 | z = 2.67 | z = −0.37 |

As is apparent from Table 18, the median value of the normalized tissue EGFR amount in patient 9 who had clinical response to EGFR antibody therapy was z=2.38 or z=2.67. Thus, in this pre-treatment gastric cancer, clinical efficacy of the EGFR-targeting agent was predicted to be high due to EGFR protein overexpression (z_{normalized tissue amount}>1.96), whereas gastric cancer in patient 2 who had no clinical response to the same therapy was predicted to have low clinical efficacy of the EGFR-targeting agent (z_{normalized tissue amount}<1.96). Thereby, the clinical validity of the MRM method, which analyzes only tumor tissue, for predicting the clinical response to the EGFR-targeting agent, was also confirmed.

### [Example 11] Prediction of Clinical Response to Targeting Agent by Tumor Purity Correction Prediction Method

For Examples (Examples 8 and 10) in which prediction of clinical response to the EGFR or ERBB2 (HER2)-targeting agent was performed only with tumor tissue isolated from pre-treatment patients, prediction became more precise through tumor purity correction of the tissue sample. As described in Example 8, both patients 7 and 8 received combination therapy with trastuzumab, which is a HER2-targeting agent, as first-line anticancer therapy for metastatic gastric cancer, but patient 7 showed partial response, and treatment in patient 8 was discontinued due to progressive disease at the time of first response evaluation, indicating good/poor clinical response results in respective cases. As described in Example 10, patient 9 received combination therapy with EGFR antibody, indicating a good clinical result of partial response. Patient 2 received combination therapy with the same EGFR antibody, but was judged to be progressive disease at the time of first response evaluation, indicating poor clinical response with no further administration.

For Examples 8 and 10 analyzed by the z value of the normalized tissue amount of the tumor sample compared to the normalized tissue amount of all-tumor MRM database, the normalized tissue amount corrected for tumor purity on H/E in 10 samples for which tumor purity information was obtained by genome analysis (NGS) on the same tissue sample was used as all-tumor MRM database and analyzed. Cases in which the median value of the housekeeping control-normalized purity-adjusted amount exceeds the standard deviation of +1.96 from the average purity-adjusted amount of all-tumor MRM database are predicted as samples with high clinical response to the relevant targeting agent.

Table 19 below shows the median z_{purity-adjusted amount} values of ERBB2 corrected for tumor purity applied in Example 8.

TABLE 19

| | Patient 7: ERBB2 | |
|---|---|---|
| | antibody therapy | Patient 8: ERBB2 antibody |
| | clinical response | therapy clinical non- |
| | tumor | response tumor |
| Median | 15.4 | 0.13 |
| z_{purity-adjusted amount} | | |
| of ERBB2_4 | | |
| Median | 19.4 | 0.28 |
| z_{purity-adjusted amount} | | |
| of ERBB2_5 | | |

As is apparent from Table 19, the median z purity-adjusted amount value in gastric cancer of patient 7 who had clinical response to the ERBB2 (HER2)-targeting agent corresponded to the inventive results predicted to have high clinical efficacy (z_{purity-adjusted amount}>1.96), whereas the median z_{purity-adjusted amount} value in gastric cancer of patient 8 who had no clinical response to the same therapy was predicted to have low clinical efficacy (z_{purity-adjusted amount}<1.96), indicating the clinical validity of the present invention for predicting clinical response. Table 20 below shows the median z_{purity-adjusted amount} values of EGFR corrected for tumor purity in MRM data of Example 10.

TABLE 20

| | Patient 9: EGFR | Patient 2: EGFR |
|---|---|---|
| | antibody therapy | antibody therapy |
| | clinical response | clinical non-response |
| | tumor | tumor |
| Tumor purity (targeted | 0.31 | 0.39 |
| DNA sequencing) | | |
| Median z_{purity-adjusted amount} | 2.64 | −0.44 |
| of EGFR_2 | | |
| Median z_{purity-adjusted amount} | 2.65 | −0.44 |
| of EGFR_4 | | |

As is apparent from Table 20, the median z_{purity-adjusted amount} value in gastric cancer (patient 2) who had no clinical response to the same therapy was predicted to have low clinical efficacy (z_{purity-adjusted amount}<1.96), whereas the median z_{purity-adjusted amount} value in gastric cancer (patient 9) who had clinical response to the EGFR-targeting agent was higher than that of Example 10 and was expected to have slightly high clinical efficacy (z_{purity-adjusted amount}>1.96), confirming the clinical validity of the present invention for predicting clinical response.

1. A method of predicting response to a cancer-targeting agent, comprising:
(a) performing multiple reaction monitoring (MRM) mass spectrometry on target peptides and housekeeping control peptides in pre-treatment cancer tissue sample and its adjacent normal tissue sample isolated from a patient using triple quadrupole mass spectrometry and nano-liquid chromatography;
(b) correcting an expression level of the target peptide measured in the multiple reaction monitoring (MRM) to a relative expression level (ratio_{tumor/normal}) in tumor tissue to normal tissue with the housekeeping control peptide using Equation 1 and Equation 2 below:
$$amount \text{?} = AUC \text{?} / AUC \text{?} \times ( amount \text{?} / \text{?} total protein ) \qquad [ Equation 1 ]$$
$$Ratio \text{?} = \frac{amount \text{?}}{amount \text{?}} \times \text{?} [ \frac{amount \text{?}}{amount \text{?}} ] ; \qquad [ Equation 2 ]$$
$$\text{?} \text{indicates text missing or illegible when filed}$$
and
(c) predicting clinical response to the targeting agent by determining a level of overexpression of the target peptide based on a corrected relative expression level (ratio_{tumor/normal}) result.

2. The method according to claim 1, wherein the sample is isolated from a pre-treatment patient before immunotherapy.

3. The method according to claim 1, wherein:
the target peptide comprises at least one selected from the group consisting of epidermal growth factor receptor (EGFR), MET proto-oncogene receptor tyrosine kinase (MET), fibroblast growth factor receptor 2 (FGFR2), erb-b2 receptor tyrosine kinase 2 (ERBB2), PD-L1 (CD274), TAP2 (TAP2), I23O1 (IDO1), SYWC (WARS1), and UB2L6 (UBE2L6), and the housekeeping control peptide is mitogen-activated protein kinase 1 (MAPK1), cyclophilin B (PPIB), and beta-actin (β-actin, ACTB).

4. The method according to claim 3, wherein:
EGFR comprises amino acid sequences of SEQ ID NO: 1 and SEQ ID NO: 2,
MET comprises amino acid sequences of SEQ ID NO: 3 and SEQ ID NO: 4,
FGFR2 comprises amino acid sequences of SEQ ID NO: 5 and SEQ ID NO: 6,
ERBB2 comprises amino acid sequences of SEQ ID NO: 7 and SEQ ID NO: 8,
PD-L1 (CD274) comprises amino acid sequences of SEQ ID NO: 9 and SEQ ID NO: 10,
TAP2 (TAP2) comprises amino acid sequences of SEQ ID NO: 11 and SEQ ID NO: 12,
I23O1 (IDO1) comprises amino acid sequences of SEQ ID NO: 13 and SEQ ID NO: 14,
SYWC (WARS1) comprises an amino acid sequence of SEQ ID NO: 15, and
UB2L6 (UBE2L6) comprises an amino acid sequence of SEQ ID NO: 16.

5. The method according to claim 3, wherein the target peptide comprises EGFR, and when a relative expression level (ratio_{tumor/normal}) of EGFR is greater than 5, clinical response to an EGFR-targeting agent when used alone or in combination in the patient is predicted to be high.

6. The method according to claim 3, wherein the target peptide comprises MET, and when a relative expression level (ratio_{tumor/normal}) of MET is greater than 5, clinical response to a MET-targeting agent when used alone or in combination in the patient is predicted to be high.

7. The method according to claim 3, wherein the target peptide comprises FGFR2, and when a relative expression level (ratio_{tumor/normal}) of FGFR2 is greater than 5, clinical response to an FGFR2-targeting agent when used alone or in combination in the patient is predicted to be high.

8. The method according to claim 3, wherein the target peptide comprises ERBB2, and when a relative expression level (ratio_{tumor/normal}) of ERBB2 is greater than 5, clinical response to a HER2-targeting agent when used alone or in combination in the patient is predicted to be high.

9. The method according to claim 3, wherein the target peptide comprises i) PD-L1 (CD274) or ii) TAP2 (TAP2), I23O1 (IDO1), SYWC (WARS1), and UB2L6 (UBE2L6), and when a relative expression level (ratio_{tumor/normal}) of PD-L1 (CD274) is greater than 3 or quantification is possible only in tumor tissue, or when an average relative expression level (ratio_{tumor/normal}) of TAP2 (TAP2) , I23O1 (IDO1), SYWC (WARS1), and UB2L6 (UBE2L6) is greater than 5, clinical response to an immunotherapy-targeting agent when used alone or in combination in the patient is predicted to be high.

10. The method according to claim 1, wherein the cancer is solid cancer.

11. The method according to claim 10, wherein the solid cancer is gastrointestinal cancer.

12. A method of predicting response to a cancer-targeting agent, comprising:
(a) performing multiple reaction monitoring (MRM) mass spectrometry on a target peptide and a housekeeping control peptide in a gastric cancer tissue sample isolated from a patient using triple quadrupole mass spectrometry and nano-liquid chromatography;
(b) obtaining a z_{normalized tissue amount} value by correcting an expression level of the target peptide measured in the multiple reaction monitoring (MRM) with the housekeeping control peptide using Equation 1 and Equation 3 below and substituting a corrected value into a database:
[Equation 1] [symbol]
[Equation 3] [symbol]
and
(c) predicting clinical response to the targeting agent by determining a level of overexpression of the target peptide based on a z_{normalized tissue amount} result.

13. The method according to claim 12, wherein:
the target peptide comprises at least one selected from the group consisting of epidermal growth factor receptor (EGFR), MET proto-oncogene receptor tyrosine kinase (MET), fibroblast growth factor receptor 2 (FGFR2), erb-b2 receptor tyrosine kinase 2 (ERBB2), PD-L1 (CD274), TAP2 (TAP2), I23O1 (IDO1), SYWC (WARS1), and UB2L6 (UBE2L6), and the housekeeping control peptide is mitogen-activated protein kinase 1 (MAPK1), cyclophilin B (PPIB), and beta-actin (β-actin, ACTB).

14. The method according to claim 13, wherein the target peptide comprises EGFR, and when a median z_{normalized tissue amount} value of EGFR is greater than 1.96, clinical response to an EGFR-targeting agent when used alone or in combination in the patient is predicted to be high.

15. The method according to claim 13, wherein the target peptide comprises MET, and when a median z_{normalized tissue amount} value of MET is greater than 1.96, clinical response to a MET-targeting agent when used alone or in combination in the patient is predicted to be high.

16. The method according to claim 13, wherein the target peptide comprises FGFR2, and when a median z_{normalized tissue amount} value of FGFR2 is greater than 1.96, clinical response to an FGFR2-targeting agent when used alone or in combination in the patient is predicted to be high.

17. The method according to claim 13, wherein the target peptide comprises ERBB2, and when a median z_{normalized tissue amount} value of ERBB2 is greater than 1.96, clinical response to a HER2 (ERBB2)-targeting agent when used alone or in combination in the patient is predicted to be high.

18. The method according to claim 13, wherein the target peptide comprises PD-L1 (CD274), and when a median z_{normalized tissue amount} value of PD-L1 (CD274) is greater than 1.96, clinical response to immunotherapy (PD-L1/PD-1 antibody) when used alone or in combination in the patient is predicted to be high.

19. The method according to claim 13, wherein a normalized tissue amount in step (b) is corrected with tumor purity using Equation 4, and a corrected value is substituted into a purity-adjusted MRM database in gastric cancer to obtain z_{purity-adjusted amount} of each of PD-L1, ERBB2, EGFR, FGFR2, and MET, and when a median value thereof is greater than 1.96, clinical response to the targeting agent when used alone or in combination is predicted to be high.


SEQUENCE LISTING (20 sequences)

SEQ ID NO: 1 - protein, 8 aa, Artificial Sequence
  EGFR_2
ITDFGLAK

SEQ ID NO: 2 - protein, 9 aa, Artificial Sequence
  EGFR_4
YLVIQGDER

SEQ ID NO: 3 - protein, 8 aa, Artificial Sequence
  MET_2
DLGSELVR

SEQ ID NO: 4 - protein, 10 aa, Artificial Sequence
  MET_4
GNDIDPEAVK

SEQ ID NO: 5 - protein, 8 aa, Artificial Sequence
  FGFR2_1
EAVTVAVK

SEQ ID NO: 6 - protein, 11 aa, Artificial Sequence
  FGFR2_2
LHAVPAANTVK

SEQ ID NO: 7 - protein, 8 aa, Artificial Sequence
  ERBB2_4
ELVSEFSR

SEQ ID NO: 8 - protein, 7 aa, Artificial Sequence
  ERBB2_5
VLQGLPR

SEQ ID NO: 9 - protein, 8 aa, Artificial Sequence
  PDL1_1
LQDAGVYR

SEQ ID NO: 10 - protein, 7 aa, Artificial Sequence
  PDL1_2
VNAPYNK

SEQ ID NO: 11 - protein, 11 aa, Artificial Sequence
  TAP2_1
AHQILVLQEGK

SEQ ID NO: 12 - protein, 10 aa, Artificial Sequence
  TAP2_2
QDLGFFQETK

SEQ ID NO: 13 - protein, 7 aa, Artificial Sequence
  I23O1_1
VIPTVFK

SEQ ID NO: 14 - protein, 11 aa, Artificial Sequence
  I23O1_2
YILIPASQQPK

SEQ ID NO: 15 - protein, 9 aa, Artificial Sequence
  SYWC
AIDQDPYFR

SEQ ID NO: 16 - protein, 13 aa, Artificial Sequence
  UB2L6
ALLLPDQPPYHLK

SEQ ID NO: 17 - protein, 9 aa, Artificial Sequence
  MAPK1_1
GQVFDVGPR

SEQ ID NO: 18 - protein, 8 aa, Artificial Sequence
  MAPK1_2
LFPNADSK

SEQ ID NO: 19 - protein, 13 aa, Artificial Sequence
  PPIB
TVDNFVALATGEK

SEQ ID NO: 20 - protein, 10 aa, Artificial Sequence
  ACTB
AGFAGDDAPR